(19) United States
(12) Patent Application Publication
(10) Pub. No.: US 2025/0303018 A1
(43) Pub. Date: Oct. 2, 2025

(54) HEMOSTATIC BIOADHESIVE

(71) Applicant: THE ROYAL INSTITUTION FOR THE ADVANCEMENT OF LEARNING/MCGILL UNIVERSITY, Montréal (CA)

(72) Inventors: Guangyu BAO, Montréal (CA); Luc MONGEAU, Mont Royal (CA); Jianyu LI, Verdun (CA); Qiman GAO, Minneapolis, MN (US); Mitchell STRONG, Beaconsfield (CA)

(21) Appl. No.: 18/866,290

(22) PCT Filed: May 25, 2023
(86) PCT No.: PCT/CA2023/050716

§ 371 Date: Nov. 15, 2024

Related U.S. Application Data
(60) Provisional application No. 63/346,027, filed on May 26, 2022.

(51) Int. Cl. A61L 24/00; A61L 24/08

(52) U.S. Cl. CPC A61L 24/0015; A61L 24/0036; A61L 24/08; A61L 2300/404; A61L 2300/418

(57) ABSTRACT

There is provided a hemostatic bioadhesive including from 0.5 to 5 w/v % of chitosan or alginate measured as a weight percent of chitosan or alginate with respect to a total dry volume of the hemostatic bioadhesive, the chitosan or alginate being crosslinked and forming a first polymer network; from 0.5 to 13 w/v % of polyacrylamide or polyethylene glycol measured as a weight percent of polyacrylamide or polyethylene glycol with respect to the total dry volume of the hemostatic bioadhesive, the polyacrylamide or polyethylene glycol being crosslinked and forming a second polymer network, wherein the first polymer network and the second polymer network form a dissipative polymer matrix; and from 0 to 100 v/v % of an adhesive liquid infused in the dissipative polymer network. The hemostatic bioadhesives comprise interconnected pores in the dissipative polymer matrix having a size of 20 to 400 μm.

### CROSS-REFERENCE TO A RELATED APPLICATION

The present application claims priority from U.S. provisional patent application 63/346,027 filed on May 26, 2022 and herewith incorporated by reference in its entirety.

### TECHNICAL FIELD

This disclosure relates to the field of hemostatic bioadhesives, particularly hemostatic bioadhesives that can reduce or halt non-compressible hemorrhage, methods of using same and methods of fabricating same.

### BACKGROUND OF THE ART

Uncontrolled hemorrhage accounts for more than 30% of trauma deaths. Despite significant research efforts, critical challenges remain for treating non-compressible and deep-narrow hemorrhages, which present rapid pressurized blood flows from wound sites. Common strategies reliant on hemostatic agents alone, such as thrombin and kaolin, to promote blood clotting are limited by slow clotting rates and coagulopathies. Alternative strategies include bioadhesive sealants that block the bleeding site physically. However, existing bioadhesives are slow and ineffective in removing the rapid pressurized blood at the interface. Moreover, situations in point-of-care and emergency rooms impose certain requirements on the properties of a bioadhesive such as being easy-to-use and having a good storage stability. Accordingly, improvements in the design and materials of bioadhesives for non-compressible hemorrhage are desired.

### SUMMARY

In one aspect, there is provided a hemostatic bioadhesive. The hemostatic bioadhesive has from 0.5 to 5 w/v % of chitosan or alginate, preferably chitosan, measured as a weight percent of chitosan or alginate with respect to a total dry volume of the hemostatic bioadhesive, the chitosan or alginate being crosslinked and forming a first polymer network. The chitosan can be present in a concentration of from 0.75 to 2.4 w/v %. The chitosan preferably has a degree of deacetylation of at least 65% and/or a molecular weight of from 50 kDa to 375 kDa. The hemostatic bioadhesive includes from 0.5 to 13 w/v % of polyacrylamide or polyethylene glycol, preferably polyacrylamide, measured as a weight percent of polyacrylamide or polyethylene glycol with respect to the total dry volume of the hemostatic bioadhesive, the polyacrylamide or polyethylene glycol being crosslinked and forming a second polymer network, where the first polymer network and the second polymer network form a dissipative polymer matrix. Preferably, the polyacrylamide is present in a concentration of 1 to 10 w/v %. The hemostatic bioadhesive includes from 0 to 100 v/v % of an adhesive liquid infused in the dissipative polymer network, measured as a volume of the adhesive liquid infused with respect to a maximum infused volume of adhesive liquid. The adhesive liquid is preferably present in a concentration of from 0 to 25 v/v %, and optionally includes chitosan. The adhesive liquid can comprise chitosan, N-(3-Dimethylaminopropyl)-N′-ethylcarbodiimide (EDC), and N-hydroxysuccin-imide (NHS). The hemostatic bioadhesives comprise interconnected pores in the dissipative polymer matrix having a size of 20 to 400 μm, 50 to 400 μm, preferably from 75 to 250 μm. The hemostatic bioadhesives optionally further comprise at least one therapeutic agent, which can be a blood clotting agent and/or an antimicrobial agent.

In one aspect there is provided, a method of reducing or stopping a hemorrhage at a bleeding site in a subject in need thereof, the method comprising covering the bleeding site with the hemostatic bioadhesive of the present disclosure, allowing the hemostatic bioadhesive to absorb interfacial fluids present at the bleeding site, and bonding the hemostatic bioadhesive to the bleeding site. The interfacial fluid comprises blood, mucus, cerebrospinal fluid, lymph fluid and/or interstitial fluid. The hemorrhage can be a non-compressible hemorrhage and the bonding can be formed without the application of external pressure.

In a further aspect, there is provided the use of the hemostatic bioadhesive of the present disclosure for reducing or stopping a hemorrhage, such as a non-compressible hemorrhage at a bleeding site in a subject in need thereof.

In yet a further aspect, there is provided a method of producing a hemostatic bioadhesive, the method comprising: mixing a solution comprising acrylamide and chitosan with a gelling solution to obtain a gel, the gel comprising 0.5 to 3 w/v % of crosslinked chitosan and 0.5 to 13 w/v % of crosslinked polyacrylamide measured as weight with respect to a total dry volume of the hemostatic bioadhesive; freeze drying the gel at a temperature of less than −2° C. to form ice crystals in the gel; and sublimating the ice crystals to leave pores having a size of from 20 to 400 μm.

In still a further aspect, there is provided a method of producing a liquid-infused microstructured bioadhesive (LIMB), the method comprising: producing the hemostatic bioadhesive according to the methods described herein; and infusing the hemostatic bioadhesive with up to 30 v/v % of a liquid measured as a volume of the adhesive liquid with respect to a total volume of the hemostatic bioadhesive.

Many further features and combinations thereof concerning the present improvements will appear to those skilled in the art following a reading of the instant disclosure.

### DESCRIPTION OF THE DRAWINGS

FIG. 1A is a schematic illustration of marine creatures that contain interconnected micropores for adhesiveness and transport of liquid reagents (prior art).

FIG. 1B is a schematic of a hemostatic bioadhesive adhering to a blood-exposed substrate.

FIG. 1C is a schematic showing that the hemostatic bioadhesive can uptake interfacial fluid, secrete functional liquids, and coagulate blood, thereby providing adhesion, hemostatic, and sealing function.

FIG. 1D is a confocal image of rhodamine-labeled LIMB containing micropores, partially filled with a FITC-labeled chitosan functional liquid.

FIG. 1E is a bar graph of the pore size of surface (1) and internal (2) pores in a liquid-infused microstructured bioadhesives (LIMB) containing 2 M or 5 M polyacrylamide (PAAm) labeled as 2M-LIMB and 5M-LIMB respectively, or 2M and 5M for brevity respectively.

FIG. 1F is a graph showing the stress-stretch curves for a LIMB containing 5 M PAAm ( [symbol] ) and a LIMB containing 2 M PAAm ( [symbol] ).

FIG. 1G is a bar graph showing the fracture energy for the 2M-LIMB and 5M-LIMB.

FIG. 1H is a bar graph showing the fractocohesive lengths for the 2M-LIMB and 5M-LIMB.

FIG. 2A is a scanning electron microscopy image (SEM) of the external surface of 2M-LIMB.

FIG. 2B is a SEM of the cross section of a 2M-LIMB showing the internal surface.

FIG. 2C is a SEM of the external surface of a LIMB containing 3 M of PAAm (3M-LIMB).

FIG. 2D is a SEM of the cross section of a 3M-LIMB showing the internal surface.

FIG. 2E is a SEM of the external surface of a 5M-LIMB.

FIG. 2F is a SEM of the cross section of a 5M-LIMB showing the internal surface.

FIG. 3A is a photograph of the experimental set up for the pure shear test before applying the shear force.

FIG. 3B is a photograph of the experimental set up of FIG. 3A when applying the shear force.

FIG. 4A is a graph of the stress in function of the strain obtained from the cyclic tensile tests with 2M-LIMB at 25 v/v % hydration.

FIG. 4B is a graph of the stress in function of the strain obtained from the cyclic tensile tests with 5M-LIMB at 25 v/v % hydration.

FIG. 5 is a bar graph showing the energy of LIMB made with varying concentrations of chitosan.

FIG. 6A is a graph of the absorption overtime showing the fluid absorption kinetics for dry LIMB (●), dry non-structured bioadhesive (NB) (▪), and hydrated NB (▴).

FIG. 6B is a graph of the adhesion energy in function of time showing the adhesion formation kinetics for LIMB with an interfacial fluid having a thickness of 250 μm (●) and 750 μm (▪), and NB with an interfacial fluid having a thickness of 250 μm (▴).

FIG. 6C is a bar graph of the effective diffusivity of NB with an interfacial fluid having a thickness of 250 μm, and LIMB with an interfacial fluid having a thickness of 250 μm and 750 μm.

FIG. 6D is a graph the frequency in function of the red intensity showing the red-signal distribution for 5M-LIMB and 2M-LIMB absorbing whole blood.

FIG. 6E is a SEM image of 2M-LIMB before absorbing whole blood.

FIG. 6F is a SEM image of 2M-LIMB after absorbing whole blood.

FIG. 6G is a bar graph showing the adhesion energy on blood-exposed surfaces for different surface pore sizes.

FIG. 6H is a graph showing the blood clotting index in function of time kinetics of whole blood placed on LIMB (●) and NB (▪). The lower the index value, the higher the degree of blood clotting.

FIG. 6I is a schematic showing different wetting scenarios of liver surface.

FIG. 6J is a bar graph showing the adhesion energy of NB and LIMB on a dry liver capsule (1) and a wet liver inside surface (parenchyma) (2) without applying any pressure.

FIG. 6K is a bar graph showing the adhesion energy of LIMB and NB on a dry liver capsule surface (1) and a wet (with blood) liver capsule surface (2). Statistical significance and p values were determined by Student t-test; ns=not significant; **=p≤0.01.

FIG. 7 is a graph of the surface pore size in function of porosity for LIMB.

FIG. 8A is a SEM image of red blood cells distributed through a cross-section of LIMB after absorption of blood.

FIG. 8B is a SEM image showing a zoom-in image of FIG. 8A.

FIG. 9A is a photograph of the adhesion of NB to an external dry liver surface (capsule).

FIG. 9B is a photograph of the non-adhesion of NB to an internal wet liver surface (parenchyma).

FIG. 9C is a photograph of the adhesion of LIMB to an external dry liver surface (capsule).

FIG. 9D is a photograph of the adhesion of LIMB to an internal wet liver surface (parenchyma).

FIG. 10A is a graph of the adhesion energy in function of the pressure showing that LIMB is pressure-insensitive at a low-pressure range. Statistical significance and p values are determined by one-way ANOVA and Tukey's multiple comparison test; ns, not significant.

FIG. 10B is a bar graph showing the adhesion of LIMB on diverse substrates with (1) and without (2) applying pressure (˜60 kPa) to form initial adhesion. The adhesion on the human aorta with pressure was not measured due to the unavailability of tissue samples. Statistical significance and p values were determined by Student t-test; ns, not significant.

FIG. 10C is a bar graph of the adhesion energy for different hydration states of LIMB.

FIG. 10D is a bar graph showing the half-life time of N-(3-Dimethylaminopropyl)-N′-ethylcarbodiimide (EDC) when stored in different adhesives.

FIG. 10E is a fluorescent microscopy image of a live/dead assay on P. aeruginosa for LIMB without benzalkonium chloride (BZK).

FIG. 10F is a fluorescent microscopy image of a live/dead assay S. aureus for LIMB without BZK.

FIG. 10G is a fluorescent microscopy image of a live/dead assay on P. aeruginosa for LIMB with BZK.

FIG. 10H is a fluorescent microscopy image of a live/dead assay S. aureus for LIMB with BZK.

FIG. 10I is a bar graph showing the quantification of the viability percentage of P. aeruginosa and S. aureus cells from the assay of FIG. 9E, when LIMB without BZK was used (1) and when LIMB with BZK was used (2).

FIG. 10J is a bar graph showing the quantification of the live bacteria per area of LIMB (i.e. attached to LIMB) of P. aeruginosa and S. aureus cells from the assay of FIG. 9E, when LIMB without BZK was used (1) and when LIMB with BZK was used (2). Statistical significance and p values were determined by Student t-test; ****p≤0.0001.

FIG. 10K is a bar graph showing the adhesion energy of LIMB infused liquid. A reservoir effect is demonstrated which enables repeatable adhesion. Statistical significance and p values are determined by one-way ANOVA and Tukey's multiple comparison test; ns, not significant; **p≤0.01.

FIG. 10L is a graph showing the adhesion in function of time of LIMB with infiltration liquid which can be loaded to and stored within LIMB at low temperature for prolonged shelf life.

FIG. 11A is a photograph showing a hemorrhage in a porcine heart.

FIG. 11B is a photograph showing the application of LIMB to the porcine heart of FIG. 11A without compression.

FIG. 11C is a photograph showing the removal of LIMB from the porcine heart of FIG. 11B.

FIG. 12A is a photograph showing a dry (0% hydration) LIMB and a wet liver parenchyma before being brought into contact.

FIG. 12B is a photograph showing the instant adhesion of dry (0% hydration) LIMB onto a wet liver parenchyma without compression.

FIG. 12C is a photograph showing the contact without compression between a fully wet (100% hydration) LIMB and a wet liver parenchyma.

FIG. 12D is a photograph showing the absence of instant adhesion of fully wet (100% hydration) LIMB with wet liver parenchyma without compression.

FIG. 13 is a graph of the adhesion energy in function of time showing the compatibility between infiltration liquid and LIMB matrix affects the activity of reagents. (Chitosan-LIMB=1, Alginate-LIMB=2, and Chitosan-NB=3). An adhesion energy above 20 J m⁻² is determined as “adhesive” and below as “not adhesive”.

FIG. 14A is a graph of the remaining hydrogel weight overtime time when exposed to phosphate buffered saline (PBS) (●) and PBS with enzymes (▪) (collagenase and lysozyme).

FIG. 14B is a fluorescent microscopy image of a live/dead staining for cells in a control environment.

FIG. 14C is a fluorescent microscopy image of a live/dead staining for cells in a in LIMB.

FIG. 14D is a graph of the viability percentage quantified from the live/dead assay of FIG. 13B for the control and LIMB.

FIG. 14E is a histology stained microscopy image 3 days NB implant (scale bar 1 mm).

FIG. 14F is a histology stained microscopy image 3 days NB implant (scale bar 100 μm).

FIG. 14G is a histology stained microscopy image 7 days NB implant (scale bar 1 mm).

FIG. 14H is a histology stained microscopy image 7 days NB implant (scale bar 100 μm).

FIG. 14I is a histology stained microscopy image 3 days LIMB implant (scale bar 1 mm).

FIG. 14J is a histology stained microscopy image 3 days LIMB implant (scale bar 100 μm).

FIG. 14K is a histology stained microscopy image 7 days LIMB implant (scale bar 1 mm).

FIG. 14L is a histology stained microscopy image 7 days LIMB implant (scale bar 100 μm).

FIG. 14M is a schematic of showing the burst pressure measurement device.

FIG. 14N is a bar graph of the burst pressure of LIMB with and without infused liquid. LIMB without infused liquid did not exhibit a noticeable sealing function while LIMB with infused liquid resisted the flow to pass through at reasonable burst pressure. Statistical significance and p values were determined by Student t-test; ***p<0.001; Sample size: N=4.

FIG. 14O is a bar graph of the burst pressure of 25%-hydrated LIMB with and without a stiff backing. Statistical significance and p values were determined by Student t-test; ***p≤0.001; Sample size: N=6.

FIG. 15A is a photograph of an implanted NB on a pig liver incision after 3 days.

FIG. 15B is a photograph of an implanted LIMB on a pig liver incision after 3 days.

FIG. 15C is a photograph of an implanted NB on a pig liver incision after 7 days.

FIG. 15D is a photograph of an implanted LIMB on a pig liver incision after 7 days.

FIG. 16A is a schematic illustration of a liver volumetric defect non-compressible hemorrhage model on rat without applying pressure.

FIG. 16B is a bar graph of the blood loss 14 days after treating the liver volumetric defect of FIG. 16A with SURGIFOAM™ or LIMB.

FIG. 16C is a bar graph showing the time of hemostasis when applying SURGIFOAM™ or LIMB to treat the liver volumetric defect of FIG. 16A.

FIG. 16D is a microscopy histology image showing a SURGIFOAM™ implant after 2-weeks (scale bar 1 mm).

FIG. 16E is a microscopy histology image showing a SURGIFOAM™ implant after 2-weeks (scale bar 200 μm).

FIG. 16F is a microscopy histology image showing a SURGIFOAM™ implant after 2-weeks (scale bar 50 μm). “Triangle” symbols point to foreign-body type giant cells; “Arrow” symbols point to lymphocytes; “Star” symbols point to eosinophils.

FIG. 16G is a microscopy histology image showing a LIMB implant after 2-weeks (scale bar 1 mm).

FIG. 16H is a microscopy histology image showing a LIMB implant after 2-weeks (scale bar 200 μm).

FIG. 16I is a microscopy histology image showing a LIMB implant after 2-weeks (scale bar 50 μm). “Triangle” symbol points to a foreign-body type giant cells.

FIG. 16J is a bargraph showing the fibrotic capsule (FC) thickness after the application of LIMB or SURGIFOAM™. Statistical significance and p values were determined by Student t-test; ns, not significant; * p≤0.5; ***p≤0.001; ****p≤0.0001.

FIG. 16K is a bar graph showing the concentration of giant cells after the application of LIMB or SURGIFOAM™. Statistical significance and p values were determined by Student t-test; ns, not significant; * p≤0.5; ***p≤0.001; ****p≤0.0001.

FIG. 16L is a bar graph showing the concentration of lymphocytes after application of LIMB or SURGIFOAM™. Statistical significance and p values were determined by Student t-test; ns, not significant; * p≤0.5; ***p≤0.001; ****p≤0.0001.

FIG. 16M is a bar graph showing the concentration of eosinophils after the application of LIMB or SURGIFOAM™. Statistical significance and p values were determined by Student t-test; ns, not significant; * p≤0.5; ***p≤0.001; ****p≤0.0001.

FIG. 16N is a schematic illustration of a deep incision hemorrhage on a rat liver.

FIG. 16O is a bar graph showing the blood loss after a deep incision on a rat liver when Gauze, Combat Gauze, NB, or LIMB is applied.

FIG. 16P is a bar graph showing the time to hemostasis after a deep incision on a rat liver when Gauze, Combat Gauze, NB, or LIMB is applied.

FIG. 16Q is a bar graph showing the adhesion strength of NB and LIMB when applied after a deep incision on a rat liver.

FIG. 16R is a schematic illustration of a deep incision hemorrhage on a pig liver.

FIG. 16S is a graph showing the blood loss of the deep incision hemorrhage on the pig liver after application of Gauze or LIMB.

FIG. 16T is a graph showing the time to hemostasis after a deep incision on a pig liver when Gauze or LIMB is applied.

FIG. 17A is a photograph showing the first part of the formation of a bleeding site having a 4-mm diameter, 3-mm deep volumetric liver injury in a rat using a biopsy punch.

FIG. 17B is a photograph showing the second part of the formation of a bleeding site having a 4-mm diameter, 3-mm deep volumetric liver injury in a rat using scissors.

FIG. 17C is a photograph showing the insertion of a hemostat into the bleeding site created as shown in FIGS. 17A and 17B.

FIG. 17D is a photograph showing the bleeding from FIG. 17C after applying SURGIFOAM™.

FIG. 17E is a photograph showing the bleeding from FIG. 17C after applying LIMB.

FIG. 18A is a photograph showing the application of Combat Gauze to a rat liver incision.

FIG. 18B is a photograph showing the bleeding after the application of Combat Gauze on a rat liver incision.

FIG. 18C is a photograph showing the application of NB to a rat liver incision.

FIG. 18D is a photograph showing the bleeding after the application of NB on a rat liver incision.

FIG. 18E is a photograph showing the application of LIMB to a rat liver incision.

FIG. 18F is a photograph showing the bleeding after the application of LIMB on a rat liver incision.

FIG. 19A is a photograph showing the marking on an incision point on a pig liver.

FIG. 19B is a photograph showing the creation of an injury on the pig liver.

FIG. 19C is a photograph showing the application of LIMB to the injured pig liver.

FIG. 20A is a schematic of the adhesion mechanism of LIMB by physical interactions.

FIG. 20B is a photograph showing the adhesion of LIMB.

FIG. 20C is a photograph of the application of a removal agent for LIMB (0.9% NaCl saline solution).

FIG. 20D is a photograph showing the removal of LIMB with the 0.9% NaCl saline solution.

FIG. 20E is a schematic of the adhesion mechanism of LIMB by covalent bonding and removal of the LIMB with acetic acid or lysozyme.

FIG. 20F is a photograph showing the adhesion of the LIMB.

FIG. 20G is a photograph showing the application of a diluted acetic acid solution to remove LIMB.

FIG. 20H is a photograph showing the instant removal of LIMB after the application of diluted acetic acid.

FIG. 20I is a photograph showing the adhesion of the LIMB.

FIG. 20J is a photograph showing the application of a lysozyme solution to remove LIMB.

FIG. 20K is a photograph showing the removal of LIMB ten minutes after the application of a lysozyme solution.

FIG. 21A is a photograph of a LIMB made with polyethylene glycol diacrylate and chitosan.

FIG. 21B is a photograph of the LIMB of FIG. 21A shown as being stretched out.

### DETAILED DESCRIPTION

There is provided a hemostatic bioadhesive for reducing or stopping hemorrhage, particularly non-compressible hemorrhage. The hemostatic bioadhesive can be a xerogel or an infused xerogel. The hemostatic bioadhesive has a polymer matrix forming the gel. More specifically, the polymer matrix is a dissipative polymer matrix formed by a double matrix. The term “dissipative” as used herein in the context of a polymer matrix means that the mechanical performance of the polymer matrix can be attributed to the dissipation of energy through hydrogen bonds and the polymer matrix has a resistance to swelling. The double matrix includes or consists of a first polymer network of chitosan or alginate, and a second polymer network of polyacrylamide or polyethylene glycol. The hemostatic adhesive can be infused with an adhesive liquid. The hemostatic bioadhesive is porous which allows the absorption of interfacial fluids such as blood, mucus, lymph, cerebrospinal fluid and interstitial fluid, which in turn grants the hemostatic adhesive an improved adhesion to wet biological surfaces. Indeed, by removing the interfacial fluids the contact surface between the hemostatic adhesive and the biological surface is increased and is less obstructed.

The first polymer network is a network formed of crosslinked alginate or chitosan. In preferred embodiments, the first polymer network is formed of crosslinked chitosan. Chitosan has better long-term stability during storage compared to alginate. This limitation of alginate hemostatic bioadhesives means that generally when an alginate-based bioadhesive is formed, it has to be used with 24 hours and cannot be store extensively. In some embodiments, the first polymer network is formed of physically crosslinked chitosan. In some embodiments, the hemostatic bioadhesive comprises from 0.5 to 5 w/v %, from 0.5 to 4 w/v %, 0.5 to 3 w/v %, from 0.5 to 2.75 w/v %, from 0.5 to 2.5 w/v %, from 0.5 to 2.4 w/v %, from 0.5 to 2 w/v %, from 0.5 to 1.5 w/v %, from 0.75 to 4 w/v %, from 0.75 to 3 w/v %, from 0.75 to 2.75 w/v %, from 0.75 to 2.4 w/v %, from 0.75 to 2 w/v %, or from 0.75 to 1.5 w/v % measured as a weight percent of chitosan or alginate with respect to a total dry volume of the hemostatic bioadhesive. The dry volume of the hemostatic bioadhesive is the volume before any liquid is infused into the hemostatic bioadhesive. The chitosan can have a degree of deacetylation (DDA) of at least 65%, at least 70%, at least 75%, at least 80%, at least 85%, at least 90% or at least 95%. Generally, a higher DDA percentage is associated with an increase in overall positive charge due to an increase in amino groups. The increase in positive charge promotes electrostatic interactions which can be desirable to improve the stability of the first polymer network as well as to improve the adhesion properties of the hemostatic bioadhesive (increase in amide bond formation with biosurfaces). In some embodiments, the chitosan has a molecular weight of 50,000 to 375,000 Da. In most cases, it was found that the molecular weight of chitosan does not have a significant impact on the performance of the adhesion.

The second polymer network is a network formed of covalently crosslinked polyacrylamide or polyethylene glycol. In some embodiments, the hemostatic adhesive comprises from 0.5 to 13 w/v %, 0.5 to 12 w/v %, from 0.5 to 10 w/v %, from 0.5 to 8 w/v %, from 0.5 to 6 w/v %, from 0.5 to 4 w/v %, from 1 to 13 w/v %, from 1 to 10 w/v %, from 1 to 8 w/v %, from 1 to 6 w/v %, from 1 to 4 w/v %, from 1.5 to 3 w/v %, or about 2.1 w/v % measured as a weight percent of polyacrylamide with respect to the total dry volume of the hemostatic bioadhesive. In some embodiments, the hemostatic adhesive comprises from 0.5 to 13 w/v %, 0.5 to 12 w/v %, from 0.5 to 10 w/v %, from 1 to 10 w/v %, from 3 to 10 w/v %, from 5 to 10 w/v %, or at least 5 w/v % % measured as a weight percent of polyethylene glycol with respect to the total dry volume of the hemostatic bioadhesive. The polyethylene glycol (PEG) preferably has a molecular weight of 5 kDa. In some embodiments, linkers can be crosslinks to PEG (for example by click crosslink with motifs such as tetrazine and norborene). In some embodiments, the concentration of polyacrylamide or PEG can be measured in molar and can be from 0.25 to 3 M, from 0.25 to 2.5 M, from 0.25 to 2 M, from 0.3 to 3 M, from 0.3 to 2.5 M, from 0.3 to 2 M, from 0.4 to 2 M, from 0.5 to 2 M, or from 0.5 to 1.5 M. In preferred embodiments, to obtain a toughness suitable for reducing or stopping a hemorrhage, the hemostatic bioadhesive comprises at least 0.3 M, preferably at least 0.4 M, more preferably at least 0.5 M of polyacrylamide or PEG. A higher chain length of polyacrylamide can improve the toughness of the adhesive matrix, and therefore contributes to higher adhesion energy. In some embodiments, the second polymer network is formed by providing a molar ratio between the acrylamide monomer to its crosslinker being 3227:1 to 13452:1.

The first polymer network and the second polymer network form a dissipative polymer matrix. The dissipative polymer matrix is a double matrix comprising the first polymer network and the second polymer network. In some embodiments, at a microscopic level, the double matrix is formed by two three-dimensional (3D) nets that interlace (a first 3D net being the first polymer network and a second 3D net being the second polymer network). There is generally no layering as the first and the second polymer network intertwine. For example, the second net can be formed by polymeric strands that go through at least a portion of the holes of the first net. On a macroscopic level the double matrix comprises or consists of a mix of chitosan or alginate, and polyacrylamide. Preferably, the double matrix comprises or consists of a mix of chitosan and polyacrylamide.

Advantageously, the hemostatic bioadhesive has pores having a size of from 20 to 400 μm, 20 to 350 μm, 20 to 300 μm, 20 to 200 μm, 50 to 400 μm, from 50 to 350 μm, from 50 to 300 μm, from 50 to 250 μm, from 50 to 200 μm, from 50 to 150 μm, from 60 to 400 μm, from 70 to 400 μm, from 80 to 400 μm, from 60 to 350 μm, from 70 to 300 μm, or from 75 to 250 μm. In some embodiments, the pores extend across a thickness of the hemostatic bioadhesive. In other words, the hemostatic bioadhesive preferably has interconnected pores. In some embodiments, the porosity is substantially uniform throughout the double polymer matrix. The porosity of the hemostatic bioadhesive does not compromise the mechanical properties and structural integrity of the hemostatic bioadhesive. Accordingly, the hemostatic bioadhesive, in some embodiments, has a double polymer matrix that has a structural integrity and mechanical properties that are not compromised or significantly affected by the porosity. In such a case, the double polymer matrix may be referred to as a pore insensitive matrix.

In some embodiments, the porosity of the double polymer matrix can be created by a cryogenic process. For example, the polymeric matrix can first be dialyzed in deionized (DI) water to remove unreacted reagents, then ice crystals can be formed through a cryogenic step. When the ice crystals melt or sublimate they leave behind an empty space and form the pores. The cryogenic step may be performed at a temperature below the freezing point of water. For example, the temperature may be less than −2° C., less than −4° C., less than −6° C., less than −10° C., less than −15° C., around −20° C., or from −10° C. to −30° C.

In other embodiments, the porosity can be created by introducing dissolvable particles or beads during the gelation of the double polymer matrix, and then dissolving the dissolvable particles or beads to create porosity in the space left behind by the dissolvable particles. In such embodiments, a drying step following the formation of the pores is generally required to dehydrate the hemostatic bioadhesives that can then optionally be infused as described herein. Compared to most alternatives, lyophilization therefore has the advantage of providing dehydrated porous hemostatic bioadhesives that can the optionally be infused as described herein.

There is generally an inverse relationship between the polyacrylamide (PAAm) or PEG concentration in the hemostatic bioadhesive and the resulting pore size. Accordingly, by reducing the concentration of PAAm or PEG the pore size can be increased. However, the formation of large craters of 1 mm or more at a surface of a bioadhesive do not constitute pores as defined in the present disclosure and provide no adhesive benefit. Without wishing to be bound by theory, large craters fail to provide adequate capillary forces to absorb the interstitial fluid and fail to provide a sufficient area of contact with a surface to obtain adequate adhesion. On the other hand, pores of a size too small are incapable of sufficiently absorbing interfacial fluid such as blood. For example, the steric interactions between small pores and blood cells can prevent pores from sufficiently absorbing blood. Inadequate small pores generally have a size of less than 50 μm or more particularly a size of less than 20 μm.

The hemostatic bioadhesive of the present disclosure combines the features of (i) a porous microstructure with pores large enough to absorb interfacial fluid and its solid components (e.g. cells and proteins), (ii) the porous microstructure is small enough to ensure adequate mechanical properties (e.g. toughness and stiffness) for the hemostatic bioadhesive, (iii) the infused adhesion liquid promotes strong interfacial bonding between the hemostatic bioadhesive and the biosurface.

The hemostatic bioadhesive also includes an adhesive liquid infused in the dissipative polymer network. In some embodiments, the adhesive liquid can be present in the hemostatic bioadhesive at from 0 to 100 v/v %, 0 to 90 v/v %, 0 to 80 v/v %, 0 to 70 v/v %, 0 to 60 v/v %, 0 to 50 v/v %, 0 to 40 v/v %, 0 to 30 v/v %, 0 to 25 v/v %, 1 to 30 v/v %, 1 to 25 v/v %, 5 to 30 v/v % or 5 to 25 v/v % as measured by a volume of the adhesive liquid infused with respect to a maximal infused volume of adhesive liquid. In other words, a 100% hydration means that the maximum of adhesive liquid that can be infused has been infused and further exposure to adhesive liquid would not significantly increase the amount of infused adhesive liquid. A 30 v/v % hydration means that the volume infused corresponds to 30% of the maximum volume that could be infused. The maximum volume can be measured with a dry bioadhesive (i.e. 0 v/v % hydration). For example, a dry bioadhesive can be submerged in a known volume of liquid and then be removed. The difference in volume between the remaining volume of liquid after removing the bioadhesive and the initial known volume can be used to determine the maximum infused volume. In general, without wishing to be bound by theory, the volume of liquid primed on the bioadhesive surface is negligible compared to the volume infused in the bioadhesive.

In some embodiments the adhesive liquid comprises a chitosan and is a chitosan-based adhesive. In some embodiments, the adhesive liquid comprises chitosan, N-(3-Dimethylaminopropyl)-N′-ethylcarbodiimide (EDC), and N-hydroxysuccin-imide (NHS), transglutaminase, oxidized polysaccharides, and/or catechol-modified biopolymers. The chitosan included in the adhesive liquid is in addition to the chitosan used in the first polymer network.

The hemostatic bioadhesive can optionally further comprise at least one therapeutic agent. The therapeutic agent may be a blood clotting agent or an antimicrobial agent. The therapeutic agent can be included in the liquid phase of the adhesive liquid and infused into the hemostatic bioadhesive along with the adhesive liquid. The hemostatic bioadhesive can optionally also comprise a storage additive to improve the shelf life of the hemostatic bioadhesive. In some cases, the therapeutic agent may also act as a storage additive.

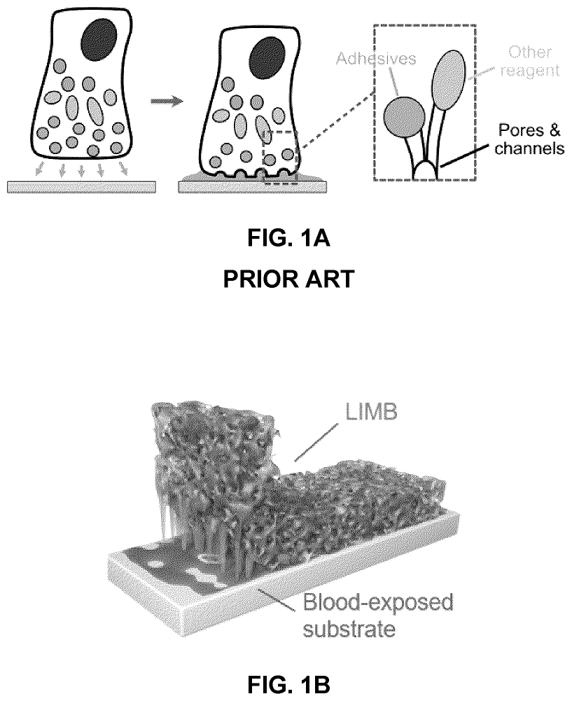

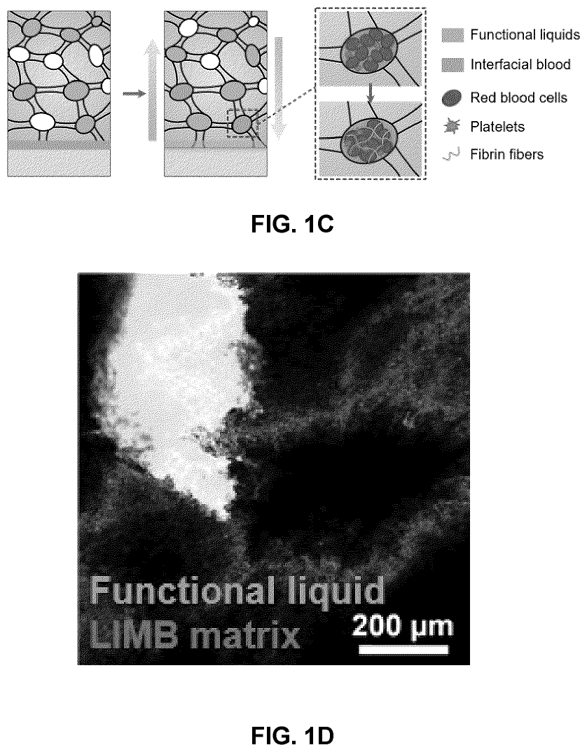

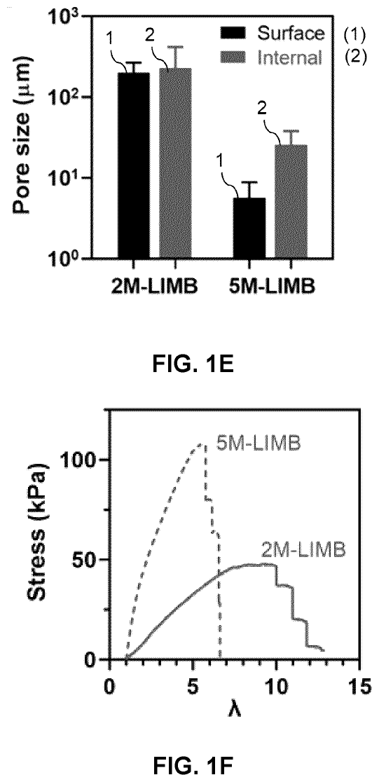

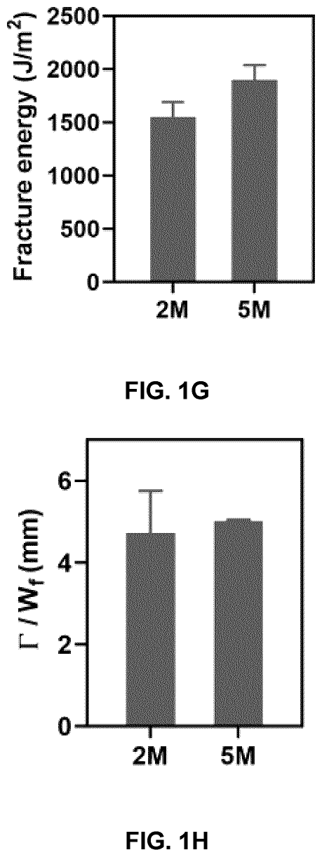

Examples of bioadhesives can be found in a multitude of living organisms. In one example, the marine environment has organisms that have developed ways to adhere to wet or “fouled” surfaces, for example mussel plaques with a microporous structure and flatworms with gland channels for storage and delivery of adhesive liquids (FIG. 1A). The hemostatic bioadhesive of the present disclosure is in contrast with traditional clinically used bioadhesives such as cyanoacrylate, fibrin glues, and hydrogel-based bioadhesives, which lack porous structures and infiltrated liquid. Catechol-based adhesives, inspired by mussels, form modest wet adhesion but do not mimic the porous structures either. Those non-structured/homogenous designs could avoid leakage and benefit sealing, but in turn, limit the ability to absorb and manipulate the interfacial fluid. Such a limitation is detrimental under hemorrhage conditions, since rapid pressurized blood can wash out hemostatic agents and disrupt any poorly-formed blood clots that are intrinsically brittle. While interfacial fluids inhibit the adhesion of materials, non-structured bioadhesives (NB) cannot rapidly remove those fluids due to the slow diffusion process and large blood components, even if a dry matrix and/or a hydrophobic repelling liquid is used. Absorbing and resisting pressurized blood flows is thus important for hemostatic technologies in treating non-compressible hemorrhage.

In some embodiments, the present disclosure provides a hemostatic bioadhesive that successfully mimics microporous structures found in nature, by producing a liquid-infused or liquid infusable microstructured bioadhesive (LIMB). The LIMB is a hemostatic bioadhesive in which the adhesive liquid or another liquid (e.g. a solution comprising the therapeutic agent) is infused therein. LIMB can rapidly absorb and clot whole blood while forming strong bioadhesion, without the need for compression, to resist blood pressure and seal bleeding sites (FIGS. 1B-1D). In some embodiments, the LIMB is formed by infusing a macroporous hemostatic xerogel with functional liquids, which differs from existing non-structured bioadhesives (NBs). LIMB is a hemostatic xerogel that is tough, biodegradable, and active in promoting blood coagulation. The pores potentiate a rapid convection and efficient removal of interfacial fluids, such as whole blood. The infused adhesive liquids enable improved and adaptable adhesiveness to different in vivo surfaces. The therapeutic agent can be provided for antibacterial function, storage stability, and easy implementation.

The hemostatic bioadhesive of the present disclosure achieves many advantages by leveraging the dissipative double network polymer matrix, tough adhesives, and liquid infiltration. The hemostatic adhesive of the present disclosure can form instant and strong adhesions with bio-fouled surfaces without the need for compression. The bioadhesives are advantageously biodegradable, easy-to-implement, and stable for long-term storage. The biodegradability is an important property for example for surgeries that require leaving the bioadhesive post-surgery to allow long term healing. With the option to infuse liquids (e.g. adhesive liquid and therapeutic agent), the functionality of the hemostatic adhesive can be tuned and optimized to a particular use case or surgical need. The hemostatic bioadhesive achieves excellent biocompatibility and hemostatic efficacy compared to several existing hemostatic agents and bioadhesives, as demonstrated by the Example section below. A further advantage of the hemostatic adhesives is that they can be instantly and safely removed after adhesion.

The adhesion of the hemostatic bioadhesive to a biosurface can be separated into two stages. In the first stage, which generally spans from contact to roughly the first two minutes following the placement, the adhesion mainly comes from the capillary suction from the dry pores of the hemostatic bioadhesive. The physical interactions can be disrupted by wetting the hemostatic bioadhesive with an aqueous phase, for example a saline solution. The hemostatic bioadhesive advantageously maintains the capacity to adhere again (re-adhesion) to the biosurface following detachment. In some embodiments, to improve the re-adhesion of a hemostatic bioadhesive, the hemostatic adhesive can be dried to reduce the volume percentage of infused liquid (e.g. interfacial fluid absorbed).

The second stage occurs after the first stage, and therefore begins around two minutes after placement of the hemostatic bioadhesive on the biosurface. The second stage generally plateaus around 10 minutes after the placement. The second stage involves the formation of chemical bonds between the hemostatic bioadhesive and the biosurface. To separate the hemostatic bioadhesive from the biosurface a chemical agent can be used to cleave the chemical bonds at the interface, for example an acidic solution (e.g. diluted acetic acid) or an enzymatic solution (e.g. lysozyme). Acetic acid can quickly disrupt the chemical bonds and to a certain degree dissolve chitosan networks at the interface and within the hemostatic adhesive, which are responsible for wet adhesion. When acetic acid is applied in vivo a neutralization step to neutralize the acid is generally required to stop the acid from affecting beyond the cleavage of the chemical bonds. In one example, a saline solution can be used to neutralize the acetic acid. Alternatively, lysozyme can act like scissors to cut down the chitosan chains and chemical bonds to thereby allow the detachment of the hemostatic bioadhesive. The detachment of the hemostatic adhesive can be performed for various reasons, for example, once the bleeding is controlled or stopped or if the hemostatic bioadhesive is misplaced. In some embodiments, the hemostatic bioadhesive promotes coagulation and when the hemostatic bioadhesive is detached, minimal or no bleeding occurs at the hemorrhage site.

Another advantage of the hemostatic bioadhesive is its ability to promote blood coagulation near and within the polymer matrix. In some embodiments, blood clotting can occur within seconds upon the contact between the hemostatic bioadhesive and whole blood. This phenomenon is unseen in non-structured bioadhesives (NB), despite the presence of the same chitosan polymer with known hemostatic function. Thus the improved blood clotting of the hemostatic bioadhesive of the present disclosure can be explained by the porosity and dehydration. Moreover, the absorption of blood into the pores concentrates RBCs and platelets, and thus accelerates substantially the clotting cascade by bringing the components closer together sterically. Besides hemostasis, the clot formation helps obstruct the pores within the hemostatic bioadhesive to avoid leakage and to improve sealing performance.

Further advantages of the hemostatic bioadhesive of the present disclosure are that the hemostatic adhesive is biodegradable, safe, biocompatible and has an improved efficacy in reducing or stopping hemorrhage, particularly non-compressible hemorrhage. In some embodiments, the hemostatic bioadhesive can be degraded by in vivo enzymes, for example lysozymes. The biodegradability is advantageous for hemostatic use to avoid the need for removal and secondary surgeries.

There are provided methods and uses of the hemostatic bioadhesive of the present disclosure for reducing or stopping the bleeding of a hemorrhage in a subject in need thereof, particularly a non-compressible hemorrhage. Examples of non-compressible hemorrhage include but are not limited to deep wounds with small entrances, for instance, caused by firearms, which limit the direct contact between hemostats and bleeding vessels. Many tissue surfaces are fouled/covered with biological substances such as mucus and blood which impair the performance of bioadhesives. For example in the case of the liver, the outer part of the liver (Glisson's capsule) may be covered with blood under traumatic and surgical conditions, while the inner part of the liver (parenchyma) is layered with a viscous interstitial fluid. The bioadhesion performance of the hemostatic bioadhesive is flexible across the different potential sites of non-compressible hemorrhage in vivo.

### Example

### Materials

All chemicals were purchased and used without further purification. The materials for hydrogel synthesis include: acrylamide (AAm, Sigma™, A9099), N,N′-methylenebisacrylamide (MBAA; Sigma-Aldrich™, M7279), ammonium persulphate (APS, Sigma-Aldrich™, A3678), tetramethylethylenediamine (TEMED, Sigma-Aldrich™, T7024), chitosan (degree of deacetylation, DDA: 95%, medium and high molecular weight, Lyphar™ Biotech), alginate (high molecular weight, 1-1G, KIMICA™ Corporation), sodium bicarbonate (Fisher™ Scientific, S233), sodium phosphate monobasic (NaH₂PO₄, Sigma-Aldrich™, S8282), sodium phosphate dibasic (Na₂HPO₄, Sigma™, S7907), acetic acid (Sigma-Aldrich™, A6283), benzalkonium chloride (BZK, Fisher™ Scientific, AA4133914). Gelatin methacrylate (GelMA) was synthesized according to a previously reported protocol (Ravanbakhsh, H. et aL. Freeform cell-laden cryobioprinting for shelf-ready tissue fabrication and storage. Matter (2021) doi:10.1016/j.matt.2021.11.020) and used as a degradable crosslinker. Materials for adhesion experiments included: N-(3-Dimethylaminopropyl)-N′-ethylcarbodiimide hydrochloride (EDC, Sigma-Aldrich™, 03450), N-hydroxysuccinimide (NHS, Sigma-Aldrich™, 130672), collagen casing (Weston™), and Very High Bond™ tape (VHB™) (3M). Porcine liver, heart, and skin tissues were purchased from a local grocery store. Materials for synthesizing fluorescently labeled hydrogels include: fluorescein-5 isothiocyanate (Thermo™ Fisher, F1907), rhodamine-B isothiocyanate (Cayman™ Chemical, 20653), anhydrous methanol (Fisher™ Scientific, A412-1), 0.22 μm PES filters (Fisher™ Scientific, 13100106), and 3.5K MWCO dialysis tubing (Fisher™ Scientific, P188244).

### Statistical Analysis

A sample size of N≥3 was used for all experiments. Data are shown as mean±SD. Statistical analysis was performed using one-way ANOVA and post hoc Tukey tests for multiple comparisons or Student's t-tests for comparison between two groups (Prism 9), p values <0.05 were considered statistically significant.

### Gel Synthesis

Both acrylamide and chitosan powders were first dissolved in 0.2 M acetic acid at 3.3 mol/L and 2.5 w/v %, respectively, yielding a solution referred to as AAm-chitosan solution. Gelatin methacrylate (GelMA) was added to the AAm-chitosan solution as the degradable crosslinker at a concentration of 0.11% w/v to obtain a precursor solution. A gelling solution to induce physical cross-linking of chitosan was prepared by first mixing 0.1M Na₂HPO₄ and 0.1M NaH₂PO₄ with a volume ratio of 50:3, followed by the addition of sodium bicarbonate to a final concentration of 0.306 M. Ammonium persulphate (APS) was added to the gelling solution at a concentration of 0.225% to act as an initiator of gelation. Both solutions were degassed, mixed at 3:2 volume ratio (precursor solution to gelling solution), and poured into a glass mold for gelation at room temperature overnight.

### Design and Synthesis of Liquid-Infused (or Infusable) Microstructured Bioadhesives (LIMB)

The design criteria of LIMB included: (i) the matrix should contain macropores (˜100 μm) that exceed the dimensions of blood components like red blood cells (6-8 μm); (ii) the matrix should be tough to tolerate the pores and dry to imbibe the interfacial fluid spontaneously; (iii) the infused liquid should facilitate strong interfacial bonding for bioadhesion and remain stable within the matrix for repetitive usage and storage. Following these design criteria, a macroporous tough xerogel was synthesized and tested as the LIMB matrix. The model xerogel was formed with covalently cross-linked polyacrylamide (PAAm) and physically cross-linked chitosan, using freeze-drying (the PAAm was crosslinked with gelatin methacrylate which is enzymatically degradable). The xerogel matrix after lyophilization was dried and partially infused with an adhesive functional liquid, comprising chitosan, N-(3-Dimethylaminopropyl)-N′-ethylcarbodiimide (EDC), and N-hydroxysuccin-imide (NHS), to facilitate amide bond formation with tissues. The products were immediately deployed or stored at −80° C. before usage.

To meet the first design criterion, the microstructure of LIMB was engineered by optimizing the polymer concentration and gelation condition. In most of the experiments, chitosan was fixed at 1.5% w/v and the PAAm concentrations were varied from 0.5 M (2.1% w/v) to 5 M (21% w/v). The resulting products were denoted as “xM-LIMB” according to x M PAAm concentration. Chitosan concentrations of 0.75% w/v and 2.4 w/v % were also produced and tested as presented in further detail below.

To produce LIMB, NB was first prepared based on the abovementioned protocol. To generate pores, NB was first dialyzed in deionized (DI) water for 1 day to remove unreacted reagents. The 2M-LIMB was completed by first placing dialyzed NB into a −20° C. freezer for 24 hours to form ice crystals and then freeze-dried. For the 5M-LIMB, the procedure was the same as the 2M-LIMB, except that the initial concentration of acrylamide in the precursor solution was 8.3M. The synthesis of rhodamine-labeled chitosan was done following the protocol described in Bao, G. et al. Triggered micropore-forming bioprinting of porous viscoelastic hydrogels. Mater. Horiz. 7, 2336-2347 (2020).

For the 0.5M-LIMB, both acrylamide and chitosan powders were first dissolved in 0.2 M acetic acid at 0.83 mol/L and 2.5%, respectively. Gelatin methacrylate (GelMA) was added to the AAm-chitosan solution at 0.11% w/v. Tetramethylethylenediamine (TEMED) was then added to the polymer solution at a concentration of 0.5%. Ammonium persulphate (APS) was dissolved in deionized (DI) water at a concentration of 0.625% to form the initiator solution. The solutions were cooled to 4° C. to slow polymerization before freezing. The solutions were then mixed at 3:2 volume ratio (precursor solution to initiator solution) and poured into a precooled (−20° C.) glass mold.

After an incubation period of 24 hours at −20° C., the gels were taken out from the mold and thawed in a precooled (4° C.) 0.306 M sodium bicarbonate solution. The gels were then first dialyzed in DI water for 1 day to remove unreacted reagents before lyophilization to form the structured hydrogel for LIMB.

### Additional LIMB Compositions

A LIMB composition with 0.75 w/v % chitosan was produced. Acrylamide (AAm) was first dissolved in 0.2 M acetic acid at 3.3 mol/L. Chitosan (DDA: 95%) was added to the AAm solution at the concentration of 1.5%. GelMA was added to the AAm-chitosan solution as the degradable crosslinker at a concentration of 0.11% w/v. A gelling solution to induce physical cross-linking of chitosan was prepared by first mixing 0.1 M Na₂HPO₄ and 0.1 M NaH₂PO₄ with a volume ratio of 50:3, followed by the addition of sodium bicarbonate to a final concentration of 0.306 M. APS was added to the gelling solution at a concentration of 0.225% as initiator. Both solutions were degassed, quickly mixed at 3:2 volume ratio (precursor solution to gelling solution), and poured into a glass mold for gelation at room temperature overnight. To generate pores, the formed hydrogel was first dialyzed in DI water for 1 day to remove unreacted reagents. LIMB was completed by first placing dialyzed hydrogel into a −20° C. freezer for 24 hours to form ice crystals and then freeze-dried. The final chitosan concentration in LIMB was 0.75 w/v % and the concentration of PAAm was 12 w/v %. This LIMB was labeled 0.75Chi-LIMB.

In a further composition, acrylamide (AAm) was first dissolved in 0.2 M acetic acid at 3.3 mol/L. Chitosan (DDA: 75%) was added to the AAm solution at the concentration of 2.5%. GelMA was added to the AAm-chitosan solution as the degradable crosslinker at a concentration of 0.11% w/v. A gelling solution to induce physical cross-linking of chitosan was prepared by first mixing 0.1 M Na₂HPO₄ and 0.1 M NaH₂PO₄ with a volume ratio of 50:3, followed by the addition of sodium bicarbonate to a final concentration of 0.306 M. APS was added to the gelling solution at a concentration of 0.225% as initiator. Both solutions were degassed, quickly mixed at 3:2 volume ratio (precursor solution to gelling solution), and poured into a glass mold for gelation at room temperature overnight. To generate pores, the formed hydrogel was first dialyzed in DI water for 1 day to remove unreacted reagents. LIMB was completed by first placing dialyzed hydrogel into a −20° C. freezer for 24 hours to form ice crystals and then freeze-dried. The final chitosan concentration in LIMB was 1.5 w/v % and the concentration of PAAm was 12 w/v %. This LIMB was labeled 1.5Chi-LIMB.

In a further composition, acrylamide (AAm) was first dissolved in 0.2 M acetic acid at 3.3 mol/L. Chitosan (DDA: 75%) was added to the AAm solution at the concentration of 4%. GelMA was added to the AAm-chitosan solution as the degradable crosslinker at a concentration of 0.11% w/v. A gelling solution to induce physical cross-linking of chitosan was prepared by first mixing 0.1 M Na₂HPO₄ and 0.1 M NaH₂PO₄ with a volume ratio of 50:3, followed by the addition of sodium bicarbonate to a final concentration of 0.306 M. APS was added to the gelling solution at a concentration of 0.225% as initiator. Both solutions were degassed, quickly mixed at 3:2 volume ratio (precursor solution to gelling solution), and poured into a glass mold for gelation at room temperature overnight. To generate pores, the formed hydrogel was first dialyzed in DI water for 1 day to remove unreacted reagents. LIMB was completed by first placing dialyzed hydrogel into a −20° C. freezer for 24 hours to form ice crystals and then freeze-dried. The final chitosan concentration in LIMB was 2.4 w/v % and the concentration of PAAm was 12 w/v %. This LIMB was labeled 2.4Chi-LIMB

### Morphology Imaging and Characterization

The porous structure of three dehydrated samples (2M-LIMB, 3M-LIMB, and 5M-LIMB) was imaged using a field emission scanning electron microscopy (SEM) (F50, FEI) under various magnifications. The dehydrated samples were coated 4 nm Pt using a high-resolution sputter coater (ACE600™, Leica) to increase surface conductivity. Pore size was analyzed by measuring 150 pores for each type of samples using the measuring tool in ImageJ™ (USA). Porosity was calculated by first transforming the SEM images into binary images and dividing the number of white pixels by the number of black pixels. To image samples after blood absorption, the samples were first dehydrated using a CO₂ supercritical point dryer (CPD030, Leica) to preserve the original morphology before SEM imaging.

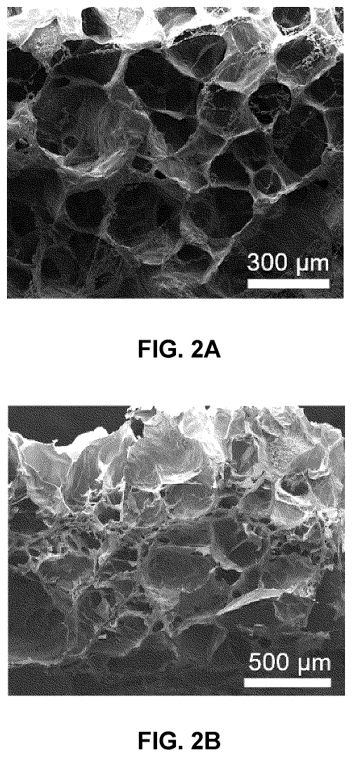

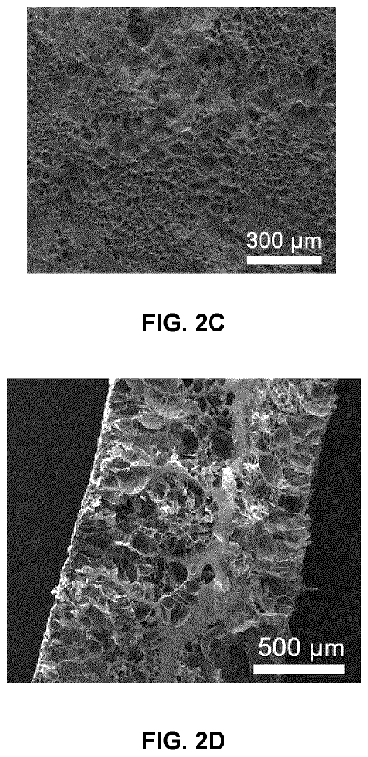

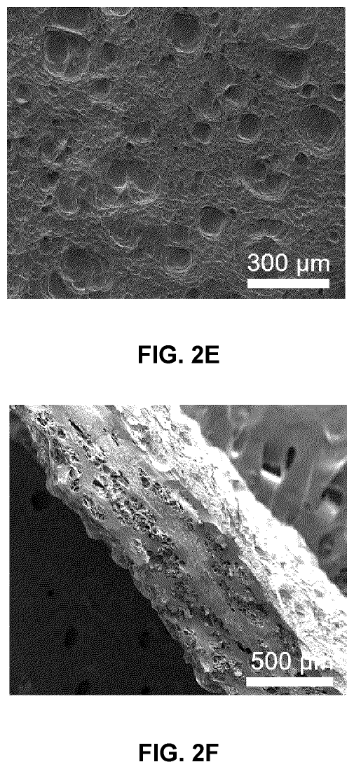

SEM imaging revealed the surfaces and internal structures of the resulting xerogels, confirming the presence of interconnected macropores within LIMB after −20° C. freezing and lyophilization (FIGS. 2A-2B). The pore size of 2M-LIMB was −200 μm and 10 to 50-fold larger than that of 5M-LIMB (FIG. 1E), indicating an inverse relationship between the PAAm concentration and the pore size. The porous structure of 2M-LIMB was uniform throughout the matrix, whereas 5M-LIMB contained much smaller surface pores than those in the bulk (FIG. 1E). Noteworthy, the cross-section view showed nonhomogeneous phase separation in the center of 5M-LIMB (FIG. 2E-2F). Results are also shown for 3M-LIMB (FIGS. 2C-2D). These findings were attributed to the high stiffness and solid content of 5M-LIMB and the fast cooling at the surface, limiting the growth of ice crystals and the resulting pore size.

### Fracture Toughness Measurement

The fracture toughness of hydrogels was measured using pure shear tests. A pair of samples (a pair of 2M-LIMB and a pair of 5M-LIMB, width W=80 mm, thickness T=1.5 mm) were glued to rigid acrylic clamps for each test. One of the pairs was unnotched, and the other pair was edge-notched. The distance between the two acrylic clamps was H=5 mm. The unnotched samples were pulled by an Instron™ machine (Model 5965) with a 1 kN load cell at a strain rate of 2 min⁻¹ to measure the stress-stretch (S-λ) curve. For the notched sample, a notch length of ˜30 mm was introduced to an edge of the sample by a razor blade. The notched samples were pulled until rupture to obtain a critical stretch (λ_{c}). The fracture energy was calculated using S-λ curve from the unnotched sample: Γ=H∫₁^{λ}^{_{c}}Sdλ.

### Tensile Test

To measure the tensile behavior under cyclic loading, the xerogels (2M-LIMB and 5M-LIMB) were cut into strips of length 35 mm, width 5 mm, and thickness 1.5 mm and tested with an Instron™ machine (10 N load cell). The displacement rate was 100 mm min⁻¹. The nominal (engineering) stress was obtained by dividing the force by the initial cross-sectional area. The nominal (engineering) strain was obtained by dividing the change in length by the original length.

### Tough and Pore-Insensitive Matrix

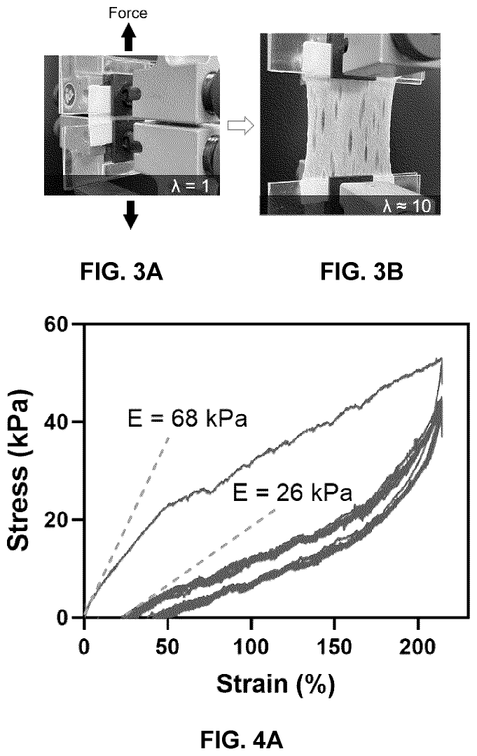

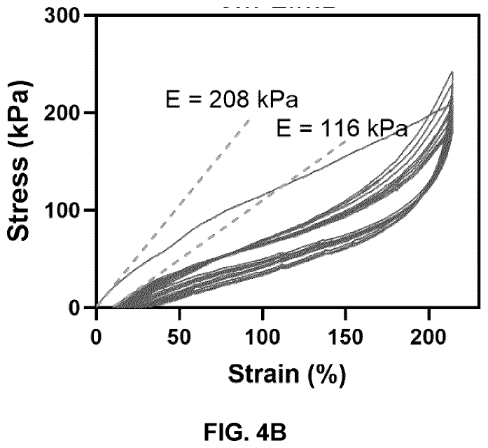

Macroporous structures are often vulnerable to rupture but this could be circumvented by a tough and pore-insensitive matrix. To test this point, pure-shear tests were performed to characterize the toughness and pore sensitivity of 2M-LIMB and 5M-LIMB. After equilibrium in phosphate-buffered saline (PBS), both LIMB samples (2M-LIMB and 5M-LIMB) exhibited high fracture energy (>1500 J m⁻²) and large deformability (stretch limit >6) (FIGS. 1F, 1G and 3A-3B). The high toughness was also confirmed with large hysteresis loops under cyclic tensile tests up to 210% strains (FIGS. 4A and 4B). The dissipative property was maintained even when the LIMBs were partially dehydrated. These properties exceeded soft tissues/organs such as cartilage and blood vessels, as well as traditional fully swollen tough adhesive. The mechanical performance of the xerogel was attributed to its double-network design, where hydrogen bonds dissipated substantial energy and resisted swelling.

To further quantify the sensitivity to pores as defects, the critical length of flaw sensitivity was estimated by dividing the fracture toughness (Γ) with the work to fracture (W_{f}, the area beneath the nominal stress-stretch curve). The critical length of LIMB (both 2M-LIMB and 5M-LIMB) was ˜5 mm (FIG. 1H), over one order of magnitude larger than the size of built-in macropores. The results implied that the LIMBs (2M-LIMB and 5M-LIMB) were immune against macropores and fracture, meeting the second design criterion.

### Adhesive Functional Liquid Preparation and Infusion

To obtain the adhesive functional liquid, 2 w/v % chitosan was first dissolved in 0.14 M acetic acid to a final pH of 5. The solution was stirred overnight before use. N-(3-Dimethylaminopropyl)-N′-ethylcarbodiimide (EDC) and N-hydroxysuccin-imide (NHS) were then added to the chitosan solution, each at 20 mg/mL. To obtain the antibacterial functional liquid, 5% benzalkonium chloride (BZK) were dissolved in water and stirred overnight to yield a clear solution at room temperature.

An important mechanical property for hemostatic bioadhesives is stiffness, which determines the conformity of a hemostatic bioadhesive to tissue surfaces. As sensitive to hydration, LIMBs (2M-LIMB and 5M-LIMB) were infused with different amounts of adhesive functional liquid and tested for Young's moduli. With 25% hydration (25% volume of LIMB was infused liquid), 2M-LIMB exhibited much lower Young's moduli (20-70 kPa) than that of 5M-LIMB (100-200 kPa) (FIGS. 4A and 4B). According to the Dahlquist criterion that soft materials are tackier when the modulus is below 100 kPa, 2M-LIMB was selected as the preferred configuration for further investigation.

### Interfacial Fluid Absorption

The swelling kinetics of LIMB, NB, and dry NB (air-dried) were measured by placing the samples on the surface of a water reservoir with only one surface in contact with the liquid. Contact time was varied from 1 to 7200 seconds at room temperature. The water absorption per sample weight was calculated by

$$\frac{{m}_{1} - {m}_{0}}{{m}_{0}} ,$$

where m₁ is the measured sample mass after absorption and m₀ the initial sample mass.

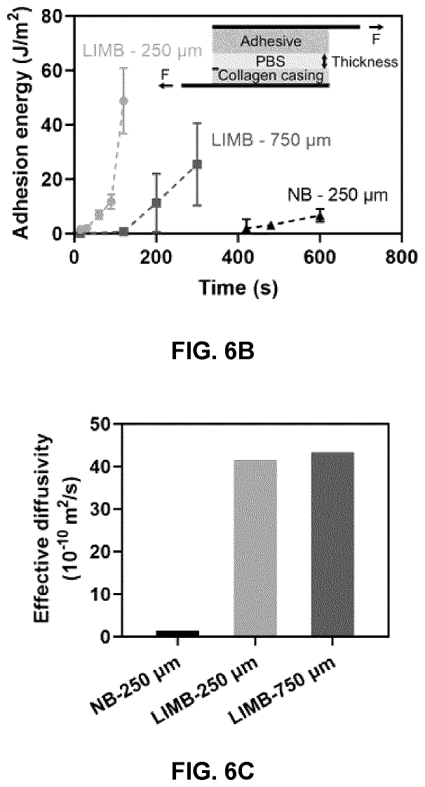

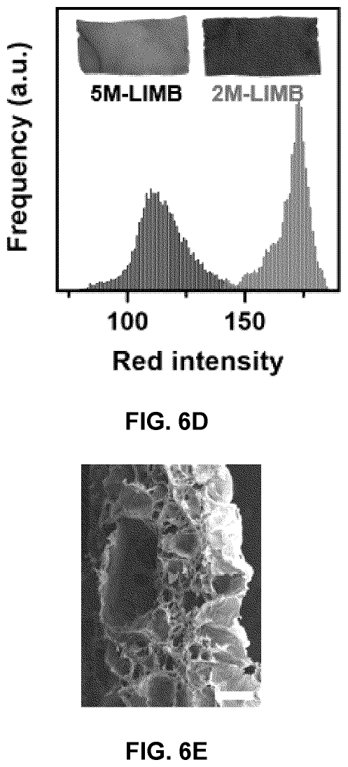

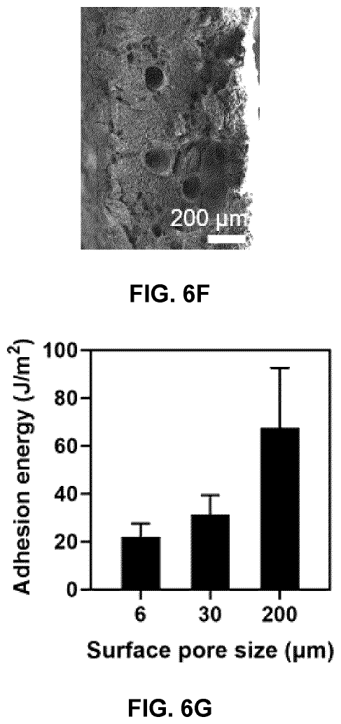

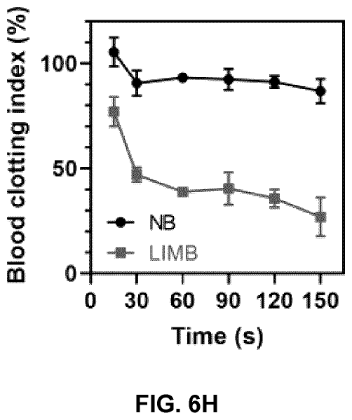

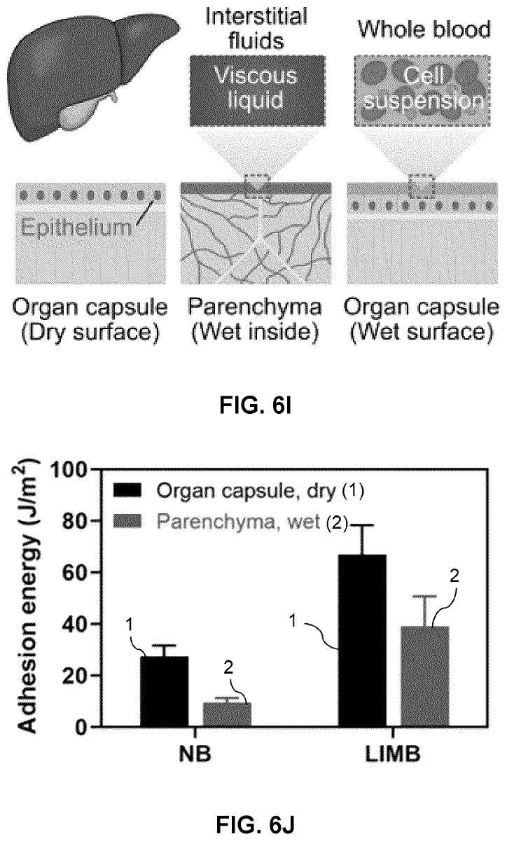

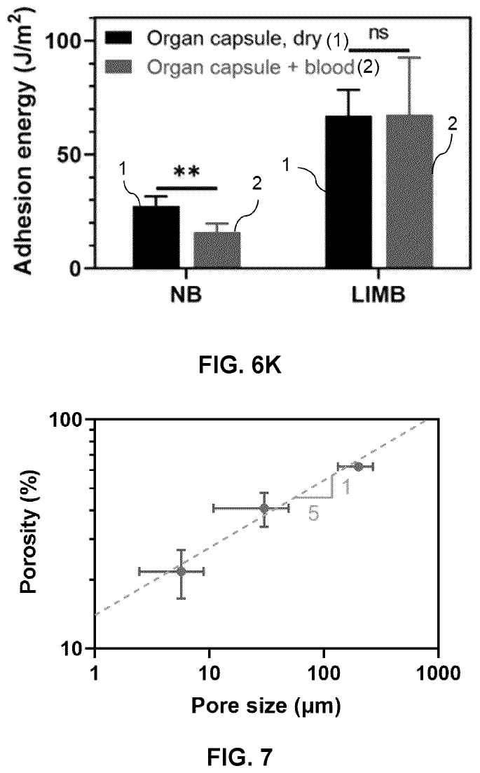

The macroporous and dehydrated nature of 2M-LIMB enabled and accelerated the uptake of interfacial fluid. The speed and capacity of interfacial fluid absorption of 2M-LIMB and non-structured bioadhesives (NBs) were characterized. 2M-LIMB absorbed liquid rapidly due to the capillary suction from the large pores compared to the diffusion-dominated mechanism of NBs at either dry or wet states (FIG. 6A). The correlation between the pore size and fluid uptake can be understood according to Washburn law. The time for 2M-LIMB to imbibe the interfacial fluid is given by:

$$t = {( \frac{h}{Φ} )}^{2} ( \frac{2 η}{γ R cos θ} ) ,$$

where h is the thickness of fluid layer, η is the viscosity of the fluid, γ is the fluid surface tension, θ is the contact angle, Φ and R are the porosity and pore size of the matrix, respectively. As seen in the measurements, Φ∝R^{1/5} (FIG. 7), the time to absorb the fluid scaled with R^{-7/5}. This indicated that the liquid uptake accelerated with larger pores, which was consistent with the results obtained herein.

### Adhesion Kinetics

The adhesion kinetics of NB, 0.75Chi-LIMB dry, 1.5Chi-LIMB dry, 2.4Chi-LIMB dry and 25%-hydrated 2M-LIMB were characterized using a modified lap-shear setup. Both the adhesive and collagen casing were firstly glued to a rigid PET (polyethylene terephthalate) backing. PBS was then evenly coated to the collagen casing on a flat surface to form a liquid barrier layer. An initial crack of 1 mm was reserved at the front of the overlapping area by attaching a piece of thin parafilm on the collagen before adding PBS. The thickness of PBS was controlled by the volume of the liquid. The adhesive with backing was then brought into contact with the collagen casing covered with the liquid barrier layer without applying any pressure. The overlapping area (width×length) of the adhesive and the collagen casing was controlled to be 20×20 mm². At a pre-determined time, the lap shear test was performed to measure the line force F (loading force/width) and nominal strain ε (displacement/length) curves. The adhesion energy was calculated using G=∫₀^{ε}^{ₘₐₓ}Fdε, where εₘₐₓ is the displacement at which F reached the maximum.

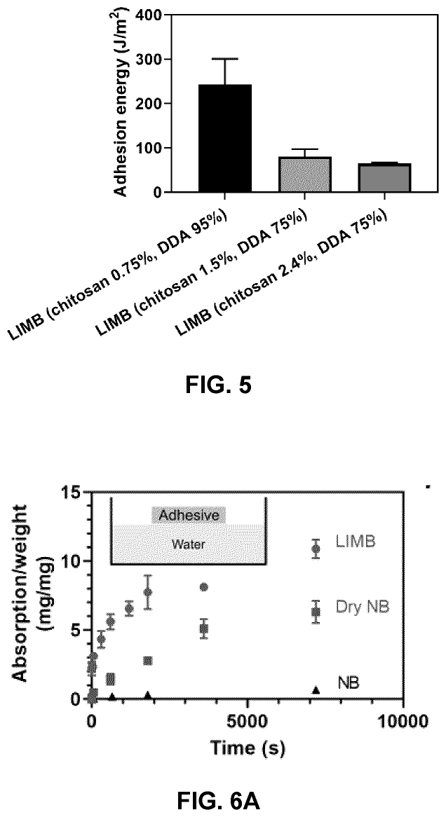

The adhesion energy for 0.75Chi-LIMB, 1.5Chi-LIMB and 2.4 Chi-LIMB were measured on a model tissue of collagen casing. The results are shown in FIG. 5.

The adhesion energy of 25% hydrated 2M-LIMB was similarly measured on collagen casing while varying the thickness of PBS at the interface. The collagen casing mimicked the tissue surface and allowed precise control over interfacial fluid to match the thickness of mucus for physiological relevance. It was found that the time to initiate adhesion (t_{ad}) was around 15 s for 25% hydrated 2M-LIMB, given the thickness of interfacial fluid (H_{if}) of ˜250 μm; when H_{if} increased to ˜750 μm, t_{ad} was prolonged to 130 s (FIG. 6B). By contrast, NB formed no adhesion after 420 s. As the adhesion was initiated upon the clean-up of the interfacial fluid, it was estimated that the effective diffusivity was (D=H_{if}²/t_{ad})˜4.2×10⁻⁹ m²/s for 25% hydrated 2M-LIMB which was 30-fold higher than that of NB (FIG. 6C).

### In Vitro Blood Clotting Assay

Clotting time assay was performed according to Quan et al. “Diaminopropionic acid reinforced graphene sponge and its use for hemostasis.” ACS applied materials & interfaces 8.12 (2016): 7666-7673 (referred to herein as Quan et al.). This assay was based on that red blood cells (RBCs) within a clot are less prone to break in hypertonic conditions compared to free RBCs due to their shape changes during clotting. 2M-LIMB, 5M-LIMB and NB were prepared in cylindrical shapes with a height of 5 mm and a diameter of 10 mm. A volume of 50 μL of recalcified human whole blood (0.2 M of 8 μL CaCl₂ added to per 100 μL human blood purchased from BioIVT) was added into the sample in a centrifuge tube. Each sample reacted with blood for 15, 30, 60, 90, 120, and 150 s. Then, 10 mL of deionized (DI) water was added to stop the reaction and to dissolve hemoglobin from free erythrocytes. A negative control comprising 50≥μL of recalcified whole blood in a centrifuge tube gave a reference value. The content of hemoglobin in the solution was measured by the absorbance of the supernatant at 540 nm using a microplate reader (Synergy™ HTX, Agilent). Six replicates were performed for each condition. The blood-clotting index (BCI) was calculated using the equation: BCI (%)=(Iₛ−I₀)/(Iᵣ−I₀), where Iₛ is the absorbance of the sample, Iᵣ is the absorbance of the reference value (negative control), and I₀ is the absorbance of DI water.

### LIMB Absorbs and Coagulates Blood

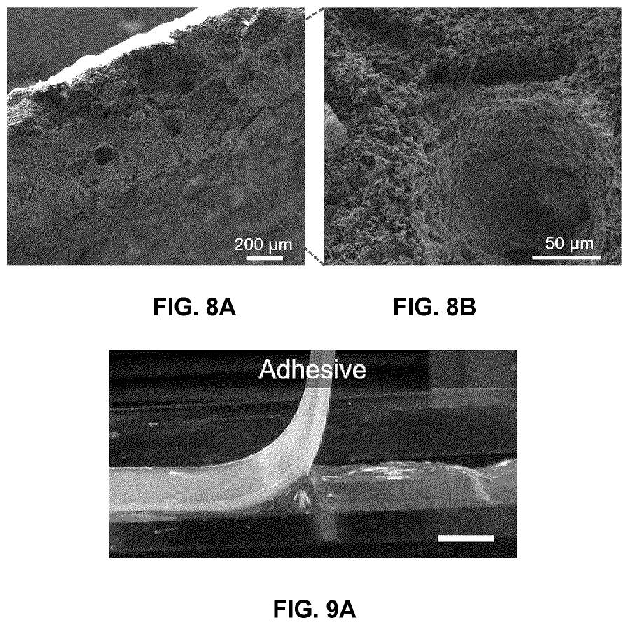

As whole blood differs from other body fluids in terms of blood cells and coagulation ability. The physical and hemostatic interactions between LIMB (2M-LIMB and 5M-LIMB) and blood were studied. First the blood absorption ability of LIMB (2M-LIMB and 5M-LIMB) was examined as a function of the surface pore size. The results showed that 2M-LIMB with larger pores absorbed the whole blood, whereas 5M-LIMB absorbed only plasma due to steric interactions between small pores and blood cells. This finding was supported by the comparison of the red signal, an indicator of red blood cells (RBCs), between the two tested conditions (FIG. 6D). SEM imaging revealed that RBCs were found in 2M-LIMB throughout the depth of the porous matrix (FIGS. 6E-6F and FIGS. 8A-8B). A downstream effect of the blood absorption was confirmed by the fact that 2M-LIMB adhered better to the liver (FIG. 6G). These results affirmed that large open pores can effectively absorb the blood cells and promote adhesion.

Another advantage of 2M-LIMB is its ability to promote blood coagulation near and within the LIMB matrix. This clotting ability was quantified with a blood clotting index, reflecting the amount of free RBCs that are not within clots, following the protocol of Quan et al. (Supra). The blood clotting index correlated inversely with the degree of blood clot formation. It was found that the clotting occurred within seconds upon the contact between 2M-LIMB and whole blood (FIG. 6H). This phenomenon is unseen in NB, despite the presence of the same chitosan polymer with known hemostatic function. Thus the difference was contributed to the porous and dehydrated nature of the 2M-LIMB matrix, which contacted and concentrated RBCs and platelets in the absorbed blood, and thus accelerated substantially the clotting cascade. Besides hemostasis, the clot formation helps obstruct the pores within 2M-LIMB to avoid transmembrane leakage for sealing performance (this is demonstrated further below). The clotting ability differentiated 2M-LIMB from the traditional bioadhesive sealants reliant solely on physical barrier effects.

### Tough Bioadhesion on Bio-Fouled Surfaces

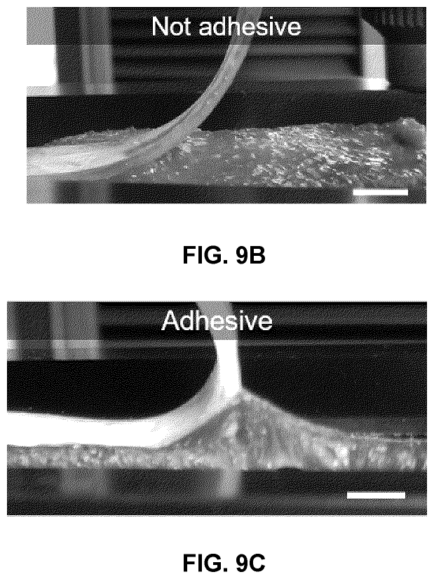

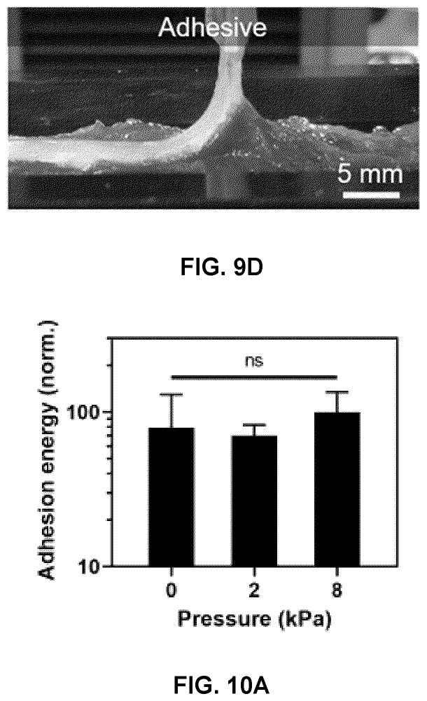

Many tissue surfaces are fouled/covered with biological substances such as mucus and blood which impair the performance of bioadhesives. For example in the case of the liver, the outer part of the liver (Glisson's capsule) may be covered with blood under traumatic and surgical conditions, while its inner part (parenchyma) is layered with a viscous interstitial fluid (FIG. 6I). The bioadhesion performance of 2M-LIMB was tested on these surfaces without compression and compared with NB (FIGS. 9A-9D). On the dry organ capsule surface, the adhesion energy of 2M-LIMB was 2.4 times higher than that of NB (FIG. 6J). Higher contrast was found on the wet parenchyma, where the adhesion energy of ˜40 J m⁻² for 2M-LIMB was more than 4 times higher than ˜9 J m⁻² for NB. Note that the weaker adhesion on parenchyma than the organ capsule was likely because the parenchyma was more fragile. A similar conclusion was found when the surfaces were fouled with blood. The performance of NB deteriorated as the blood components impair the contact between NB and the tissue. No such reduction was observed with 2M-LIMB since it effectively absorbed and removed the blood as shown in FIG. 6K.

### Alginate Xerogel Synthesis

To synthesize alginate-based xerogel, both acrylamide and alginate powders were first dissolved in water at 2 mol/L and 2.256%, respectively. N,N′-methylenebisacrylamide (MBAA) was added to the AAm-alginate solution at 0.0006:1 the weight of acrylamide to form the polymer precursor solution. A cross-linking solution containing 6.87% CaSO₄ and 3.58% ammonium persulphate (APS) was prepared in deionized water (DI). Both solutions were degassed. MBAA (weight ratio: 0.0028:1 to acrylamide) was then added to the degassed AAm-alginate precursor solution before mixing. Both solutions were mixed with a 24:1 volume ratio (polymer solution to cross-linking solution) and poured into a glass mold. The cross-linked hydrogel was then transferred to a −20° C. freezer for 24 hours and then lyophilized for at least 48 hours to form alginate xerogel.

### Adhesion Test

Various adhesives (gelatin, poly (hydroxyethyl methacrylate) (PHEMA), Very High Bond™ (VHB™) tape, PAAm alginate-PAAm) and substrates (pig liver, human aorta, pig skin, and pig heart) were cut into strips of length 80 mm, width 15 mm, and thickness 1.5 mm. Rabbit whole blood of 150 μL was dispensed evenly on the substrates and adhesives. The adhesives and substrates were brought into contact with 2M-LIMB, incubated for 30 mins at either room temperature or 4° C. in a sealed container, and then tested with peeling tests using an Instron™ machine (10 N or 1 kN load cell). Rigid polyethylene terephthalate (PET) films were glued to the back of the substrate and the adhesives, respectively, using superglue (Krazy Glue) before testing. For 90° peeling tests, the displacement rate was 50 mm min⁻¹. The adhesion energy of the specimen was calculated by dividing two times the average force at the plateau (F_{avg}) by the thickness of the specimen: Γ=F_{avg}/W. For T-peeling tests, the displacement rate was 100 mm min⁻¹. The adhesion energy of the specimen was calculated by dividing two times F_{avg} by the thickness of the specimen: Γ=2F_{avg}/W.

For adhesion tests involving human aorta, a total of 4 descending thoracic aorta samples were extracted from two organ donors. One donor was a 66-year-old male having a weight of 86 kg and a height of 162 cm. The cause of death was head trauma. The donor was healthy and with no disease. The other donor was a 47-year-old male having a weight of 92 kg and a height of 188 cm. The cause of death was stroke.

### Pressure-Insensitive Adhesion to Diverse Materials

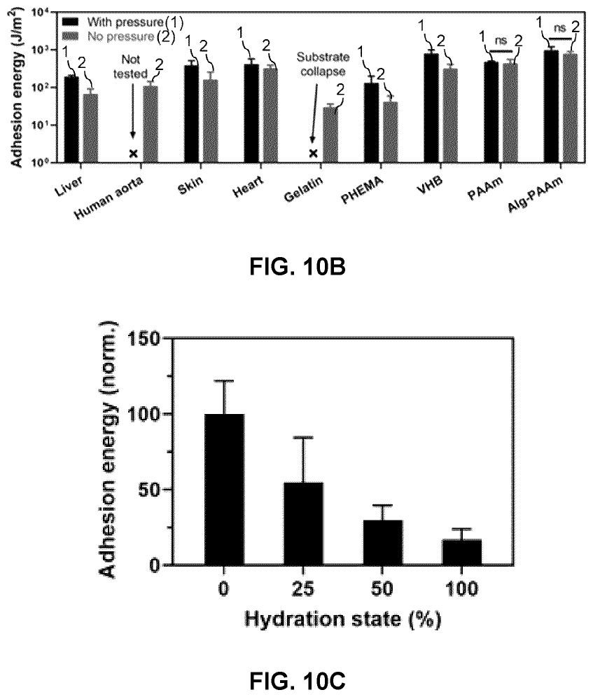

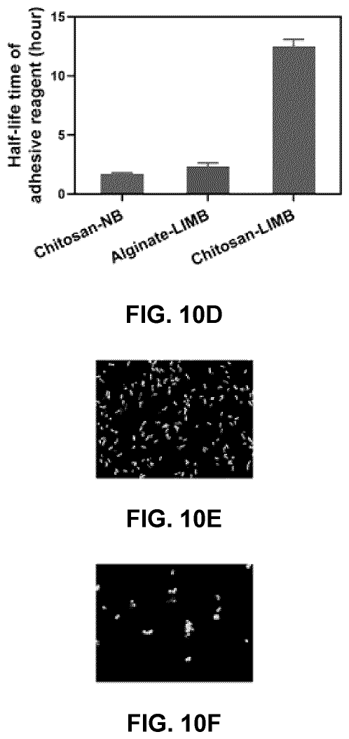

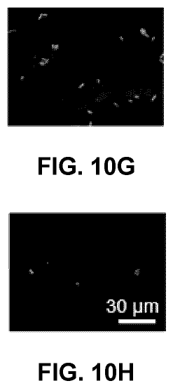

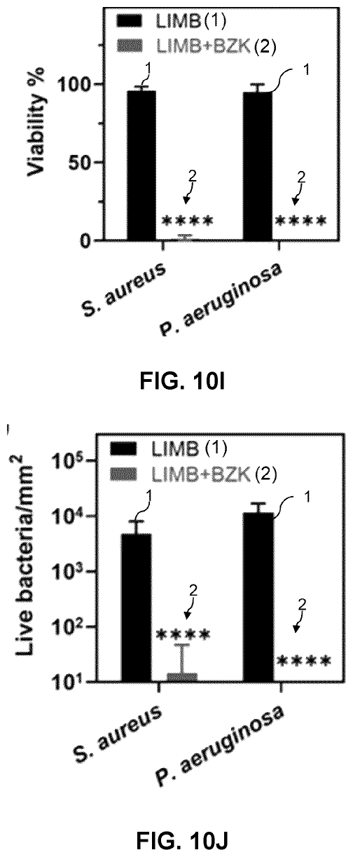

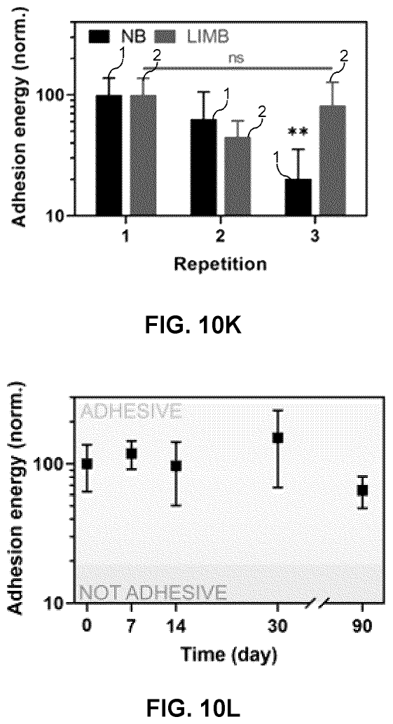

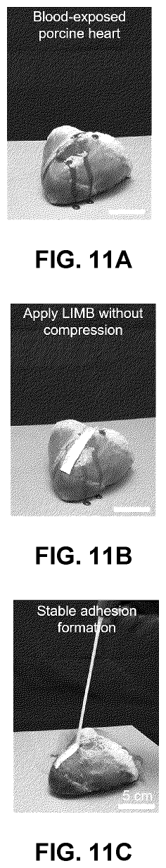

Besides the liver, 2M-LIMB achieved strong and universal adhesion without compression on diverse surfaces. For instance, the adhesion between 2M-LIMB and porcine skin was found to be independent of applied pressure (0-8 kPa) (FIG. 10A). Similar adhesion performance was found on other tissues such as human aorta and porcine heart, with or without pressure (FIG. 10B). In FIG. 10B, the bars labeled as liver (i.e. pig liver), human aorta, skin (i.e. pig skin), and heart (i.e. pig heart) were contacted with 2M-LIMB. As a demonstration, 2M-LIMB formed stable adhesion to a blood-exposed porcine heart without the need for compression (FIGS. 11A-11C). In addition to tissues, 2M-LIMB exhibited pressure-insensitive adhesion on hydrogels and elastomers, namely gelatin, PHEMA, VHB™ tape, PAAm, and alginate-PAAm (FIG. 10B). Without the pressure, 2M-LIMB still achieved a strong adhesion that outperformed fibrin glues and medical tapes (10-20 J m⁻²). Among the tested hydrogels, gelatin hydrogels were too fragile to survive the compression for adhesion formation. The robust pressure-insensitive adhesion was attributed to the unique structure of 2M-LIMB: the xerogel matrix absorbed spontaneously interfacial fluids, forming intimate contact and strong bonding with the substrates with the aid of the infused liquid. This feature enabled a wide utility of 2M-LIMB in diverse medical and engineering settings.

### Bacterial Culture and Viability Assay

Gram-negative Pseudomonas aeruginosa (PAO1) and the Gram-positive bacteria Staphylococcus aureus (ATCC 25923) were used as model bacterial strains in this study. Fresh bacterial cultures were first prepared on nutrient agar from −80° C. glycerol stocks. Bacterial suspensions were then prepared by transferring single colonies from agar plates to Luria-Bertani broth and incubating the media at 37° C. and 200 rpm (agitated plate). Once the bacteria reached the exponential growth phase, they were harvested and centrifuged at 4000 g (Thermo Scientific, Heraeus Multifuge X3R). After discarding the supernatant, the cells were suspended in PBS. The optical density of bacteria at 600 nm (OD600) was set to 0.2 using a UV spectrophotometer (Thermo Scientific, Biomate 3S). The antimicrobial property was assessed by incubating the suspended cells in 2 mL bacterial suspensions in a 12-well microtiter plate for 30 minutes. Pristine 2M-LIMB and 2M-LIMB infused with 5% BZK solution in water were tested. The samples were gently rinsed with fresh PBS afterward to remove the loosely attached cells. The cells that remained attached to the samples were then assayed for viability by adding BacLight™ stains (Molecular Probes) prepared according to the manufacturer's protocol. The BacLight™ kit contains SYTO 9, which produces fluorescent green color (excitation 483 nm/emission 503 nm) in cells with intact membranes (live cells), and propidium iodide, which gives fluorescent red color (excitation 535/emission 617 nm) to cells with compromised membranes (dead/dying cells). After 15 min incubation in the presence of stains, the samples were observed under a confocal laser scanning microscope (Zeiss™, LSM710) and at least 15 images were acquired for each substrate at various locations. The viability percentage of bacteria was calculated by dividing the number of live cells by the total number of cells.

Tunable Functionality with Infused Liquid

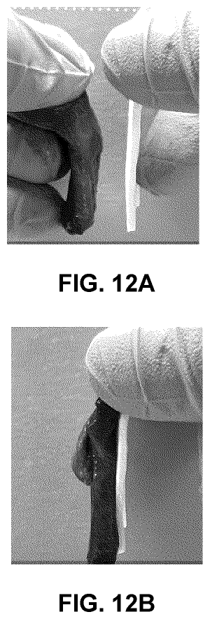

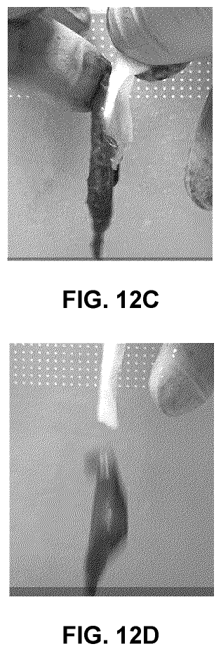

The performance and functionality of 2M-LIMB are tunable with the infused liquid. It was hypothesized that the amount of infused liquid or the hydration state could mediate the adhesion performance of 2M-LIMB. 2M-LIMB was loaded and tested with different amounts of the adhesive functional liquid (0-100%) on wet porcine heart (FIG. 10C). For 0%-hydrated 2M-LIMB, the surface of initially dry 2M-LIMB was primed with the adhesive functional liquid and immediately applied onto the tissue. The adhesion energy scaled inversely with the hydration state (FIG. 10C). A similar correlation was found between the adhesion formation speed and the hydration state, where 0%-hydrated 2M-LIMB form instant adhesion with wet tissues upon contact while 100%-hydrated 2M-LIMB did not (FIGS. 12A-12D). However, it was found that 100 v/v % hydrated bioadhesive achieved an adhesion energy above 20 J m⁻² which is considered the threshold for being adhesive.

The infused liquid can also functionalize 2M-LIMB. This was demonstrated by loading 2M-LIMB with an antimicrobial agent, 5% benzalkonium chloride (BZK), for an antibacterial function that is desired in many surgical settings (FIGS. 10E-10H). The antimicrobial and antifouling functions were tested by exposing BZK-laden 2M-LIMB to two model pathogenic bacteria: P. aeruginosa and S. aureus. Compared to the pristine 2M-LIMB, BZK-2M-LIMB exhibited more prominent bacteria-killing and attachment inhibition effects (FIGS. 10I and 10J). The results demonstrated the tunability and modularity of 2M-LIMB with the liquid-infiltration design.

### Ease of Use and Stability of Long-Term Storage.

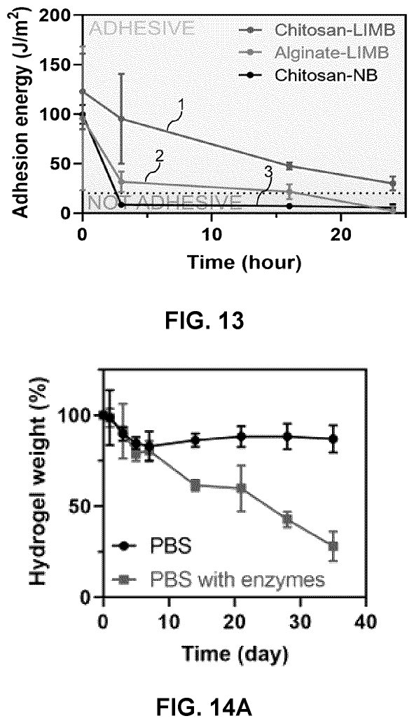

The unique design of 2M-LIMB also helps address important translational considerations about usability and storage. Minimizing chemical reactions between the liquid and the matrix is a premise for extending the time window for usage and prolonging the stability of 2M-LIMB. To do so, both the matrix and the infused liquid of 2M-LIMB were chosen to be based on chitosan (primary amine-rich); as such, no carbodiimide reaction would occur. Another LIMB was prepared made from alginate (carboxylic acid-rich) for comparison. The chitosan- and alginate-based LIMBs were loaded with the same adhesive functional liquid (chitosan, EDC and NHS) and stored at 4° C. in sealed containers. The adhesion energy of LIMBs on porcine skin was characterized as a function of storage duration. The chitosan-based 2M-LIMB maintained adhesiveness even after 24 hours (FIG. 13); the kinetics were related to the intrinsic hydrolysis of EDC. In comparison, the alginate-based LIMB exhibited a much shorter half-life time, as the infused liquid reacted with the alginate-based matrix via carbodiimide reactions (FIG. 10D); for NB, the loss of adhesiveness was attributed to the shortage of adhesive liquids.

With the confirmed compatibility, 2M-LIMB was anticipated to maintain adhesiveness during repositioning and long-term storage. On one hand, the adhesive agents stored in macropores can replenish the surface for repeatable adhesion. The adhesion energy was measured by attaching the same 50%-hydrated 2M-LIMB to fresh porcine livers three successive times, mimicking the repositioning scenario. The adhesion performance of LIMB was nearly invariant in all repetitions, whereas the adhesion energy of NB at the third attempt decreased to one-sixth of that at the first attempt (FIG. 10K). The salient feature allowed the corrections of the location of 2M-LIMB for optimal placement. On the other hand, 2M-LIMB can be stored for an extended period at −80° C., commonly used for the storage of therapeutics and chemicals. The low temperature can inhibit the degradation of the adhesive agents and further improve their stability within 2M-LIMB. To test this possibility, the adhesion performance of 25%-hydrated 2M-LIMB was determined after storage on blood-exposed liver capsules without applying compression. It was found that 25% hydrated 2M-LIMB remained highly adhesive over 90 days (FIG. 10L). These collective attributes support the excellent usability of hydrated 2M-LIMB.

### Cytocompatibility

The cytotoxicity of 2M-LIMB to immortalized human vocal fold fibroblasts was evaluated following standard protocols as described in International Organization for Standardization (ISO) 10993-12. 2M-LIMB extracts were prepared by adding 1 mL of Dulbecco's Modified Eagle's medium (DMEM) to 200 mg of 2M-LIMB in 12-well plates to completely cover the hydrogels. On the same day, in parallel, cells were seeded into 96-well plates with a density of 50,000 cells per well. After 24 hours of incubation, the extracts were collected and supplemented with 1% nonessential amino acid, 1% penicillin-streptomycin, and 10% fetal bovine serum. Then, the cell culture medium within the 96-well plate was removed and replaced with the supplemented hydrogel extracts (2M-LIMB or NB). Supplemented pristine DMEM was used as control. The treated cells were incubated at 37° C., 95% relative humidity, and 5% CO₂ atmosphere. After 24 hours, the cell viability was determined using a Live/Dead viability kit (Invitrogen, L3224) according to the manufacturer's protocol. The stained cells were visualized using a confocal laser scanning microscope (Zeiss, LSM710). Live cells were shown in green fluorescence and dead cells were shown in red.

### In Vitro Biodegradation

All samples (2M-LIMB and NB) were prepared with the same size and weighed at Day 0. After that, an enzyme solution consisting of 50 μg/mL of collagenase (MP Biomedicals™, 1951091), 50 μg/mL lysozyme (MP Biomedicals, 100831), and 0.01% sodium azide (NaN₃, Sigma, S2002) in PBS was added to the gels. The samples were incubated at 37° C. with gentle mechanical stimulation at 75 rpm over 35 days. The enzyme solution was changed every other day. At pre-determined time intervals, the enzyme solution was removed. The samples were then washed three times (5 minutes each wash) with deionized water. The samples were then lyophilized and the remaining polymer dry weight was measured. The remaining ratio of the polymer was calculated by dividing the dry weight of the remaining polymer by the dry weight of the initial gels.

### In Vitro Biodegradation and Cytocompatibility

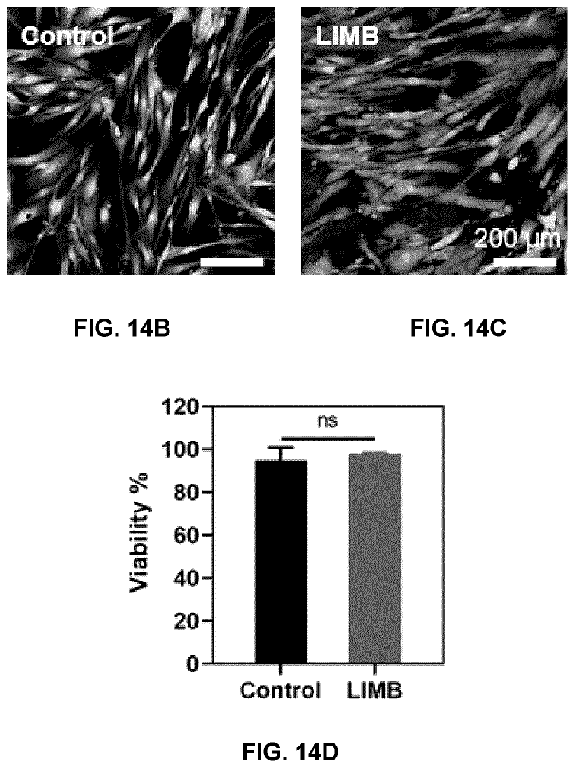

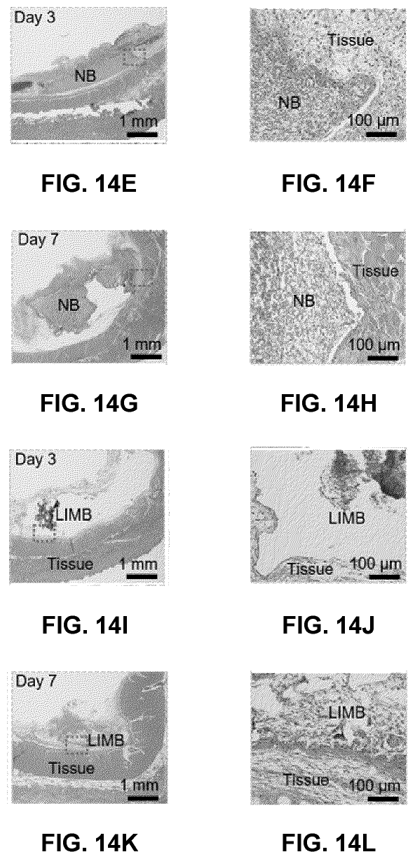

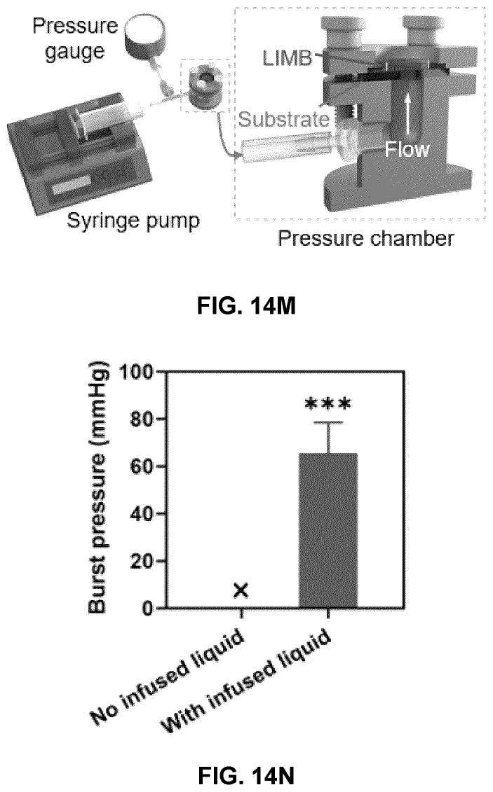

A series of in vitro and in vivo tests were combined to evaluate the safety and efficacy of 2M-LIMB for hemorrhage control. The incorporation of a degradable cross-linker for PAAm network and the biodegradable chitosan rendered 2M-LIMB biodegradable. A steady degradation profile of 2M-LIMB when exposed to an enzyme solution comprising lysozyme and collagenase at physiological levels is shown in FIG. 14A. The biodegradability is important for hemostatic use to avoid the need for removal and secondary surgeries. The cytocompatibility of 2M-LIMB was also evaluated according to the International Organization for Standardization (ISO) 10993-12: Tests for in vitro cytotoxicity standard. Immortalized human vocal fold fibroblasts (hVFFs) were used. The in vitro cell culture showed over 98% cell viability after 24 hours, proving the cytocompatibility of 2M-LIMB (FIGS. 14B-14D).

### Histology and Analysis

Fixed tissue samples were placed into 70% ethanol and submitted for histological processing and H&E staining at the Histology Core at McGill University. Z.-H. G., the pathologist-in-chief at the University of British Columbia, examined all histological sections.

### Biocompatibility

This experiment was approved by the McGill University Animal Care Committee (Protocol #2019-8098) and performed according to the guidelines of the Canadian Council on Animal Care. Female Sprague Dawley rats (250-300 g) were used for all the in vivo rat studies. Prior to implantation, both NB and 2M-LIMB were prepared into disk shape using aseptic techniques. The size was 5 mm in diameter and 2 mm in thickness. For implantation in the dorsal subcutaneous space, rats were anesthetized using isoflurane (4% isoflurane in oxygen) in an induction chamber. Anesthesia was maintained at 2% isoflurane using a nose cone during the surgery. A volume of 1 mL of saline was injected subcutaneously. Hair was removed and the rats were placed over a heating pad for the duration of the surgery. The subcutaneous space was accessed by a 1 cm skin incision per implant in the rat's back. Blunt dissection was performed from the incision point towards the shoulder blades of the rat to create subcutaneous space for implantation. NB and 2M-LIMB were implanted into the subcutaneous spaces. Up to four implants were placed per rat. The skin incisions were closed with sutures. On Day 3 and 7, rats were euthanized by 5% isoflurane induction followed by CO₂ inhalation. Subcutaneous regions of interest were excised and fixed in 4% paraformaldehyde solution for 48 hours for histological analysis.

### In Vivo Biocompatibility

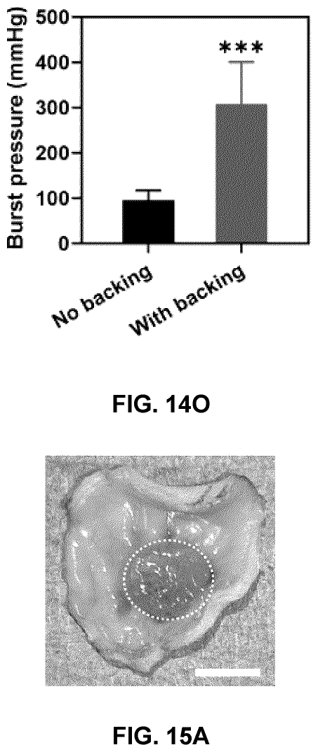

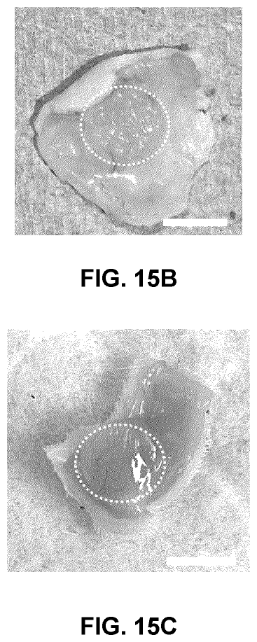

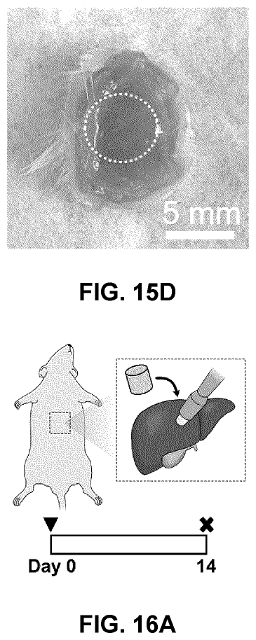

The in vivo biocompatibility of the 2M-LIMB was examined via subcutaneous implantation in rats for 7 days and compared with NB. The explants maintain physical integrity after the implantation (FIGS. 15A-15D). Histological sections showed a very thin capsule with rare foreign body-type giant cells at the interface between the tissues and all the implants (FIGS. 14E-14L). There was no evidence of active or chronic inflammation or allergic eosinophilic responses. Consistent with the cytocompatibility test, the results conclude that 2M-LIMB and NB elicited comparable minimum inflammation responses in vivo. The toxicity of EDC was dependent on concentration. When used at low concentrations, EDC was found to induce no severe toxicity (Table 1). Considering the strong adhesion and sealing effect from EDC, its benefits in treating life-threatening situations outweigh its local cytotoxicity. It is also worth noting that EDC can be replaced by other bioadhesive agents, such as transglutaminase, oxidized polysaccharides, and catechol-modified biopolymers, which are biocompatible and capable of bonding 2M-LIMB with biological tissues. 2M-LIMB as a platform technology can be customized to accommodate a variety of adhesive agents.

TABLE 1
In vivo evaluation for the toxicity of EDC-based bioadhesives

| | EDC | Animal | Implantation | Implantation | |
|---|---|---|---|---|---|
| | conc. | species | time | site | Toxicity |
| 2M-LIMB with 1.5 w/v % | 20 | Rat | 2 | Subcutaneous | Very mild immune |
| chitosan | mg/mL | | weeks | and liver | response |
| Polyacrylamide + alginate (no | 40 | Rat | 2 | Subcutaneous, | Low cytotoxicity; |
| pores i.e. 0% porosity) | mg/mL | | weeks | heart, and | Mild to moderate |
| (Science, 2017. 357: 378-381) | | | | liver | immune response; |
| | | | | | Lower toxicity |
| | | | | | compared to |
| | | | | | commercial |
| | | | | | products, such as |
| | | | | | SURGIFLOW |
| | | | | | (Ethicon), CoSeal |
| | | | | | (Baxter), and |
| | | | | | cyanoacrylate. |
| Polyacrylamide + alginate (no | 12 | Mouse | 16 | Subcutaneous | Mild immune |
| pores i.e. 0% porosity) | mg/mL | | weeks | and skin | response; No |
| (Advanced Materials, 2021. 33: | | | | | systematic |
| 2008553) | | | | | toxicity |
| Polyacrylamide + alginate (no | 12 | Rat | 3 | Tendon | No toxicity |
| pores i.e. 0% porosity) (Nature | mg/mL | | weeks | | reported |
| Biomedical Engineering, 2022. | | | | | |
| 10.1038/s41551-021-00810-0) | | | | | |
| Polyacrylamide + alginate (no | 2.4 | Rat and | 28 days | Skin (rat) | No toxicity |
| pores i.e. 0% porosity) | mg/mL | pig | (rat) | Liver | reported |
| (Bioactive Materials, 2022. 13: | | | 8 days | (pig) | |
| 260-268) | | | (pig) | | |
| Poly(N-isopropylacrylamide) + | 12 | Mouse | 1 week | Skin | Mild to moderate |
| alginate (no pores i.e. 0% | mg/mL | | | | immune response |
| porosity) | | | | | |
| (Science Advances, 2019. 5: | | | | | |
| eaaw3963) | | | | | |

### Burst Pressure Test

A burst chamber was manufactured according to the ASTM F2392: Standard test method for burst strength of surgical sealants. Cardiac tissues were harvested from the surface of a porcine heart and trimmed into circles (30 mm in diameter) with a thickness of ca. 2 mm. A biopsy punch was used to create a 3 mm diameter hole in the center of each circular tissue sample. LIMBs were cylindrical with 15 mm in diameter and 1.5 mm in thickness. For patches with backings, a circular polyethylene terephthalate (PET) film (100 μm in thickness) was attached to one side of the patches using super glue. To attach the hydrogel patches to the defected tissues, a chitosan bridging polymer containing EDC and NHS (each at 20 mg/mL) was applied to the hydrogel side. 2M-LIMB and defected tissues were brought into contact without applying compression. The specimens were kept inside a humidified container at 4° C. overnight before testing. During the test, a water-laden syringe was connected to both the burst chamber and a pressure gauge. A syringe pump was used to feed the water to the chamber. The peak pressure before either the adhesive or the cohesive failure was recorded and used as the burst pressure.

### In Vitro Sealing Test

The combination of hemostatic and bioadhesion performance allows 2M-LIMB to seal the lateral and transmembrane flows of body fluids. While the macroporous structure concerns leakage, the infiltration of the adhesive functional liquid within 2M-LIMB provided liquid gating effects to ensure sealing. To demonstrate this, the burst pressure of 2M-LIMB was measured as a function of liquid infiltration. When 25% of the pores were infused with liquid (25%-2M-LIMB), the 25%-2M-LIMB exhibited high burst pressure at ˜66 mmHg, in contrast to almost no sealing from 2M-LIMB without infused liquid (FIGS. 14M and 14N). The decent sealing was attributed to the infused liquid that closed the macropores for blood leakage. When tested on heart tissues, the 25%-2M-LIMB achieved ˜95 and ˜308 mmHg of burst pressure with and without rigid backing support, respectively, showing an excellent sealing performance (FIG. 14O). The sealing was prolonged and the attachment to the tissue persisted even after being immersed under PBS for over 8 days.

### Rat Liver Puncture Bleeding Model

For hemostatic sealing of the volumetric hepatic injury, the rats were anesthetized using isoflurane (4% isoflurane in oxygen) in an induction chamber. Anesthesia was maintained at 2% isoflurane using a nose cone during the surgery. A volume of 1 mL of saline was injected subcutaneously. Abdominal hair was removed, and the rats were placed over a heating pad for the duration of the surgery. The liver was exposed via a laparotomy. A volumetric injury of 4 mm diameter and 3 mm depth was made to the liver using a biopsy punch. Cylindrical-shaped SURGIFOAM™ (Ethicon) or 2M-LIMB of 5 mm diameter and 4 mm depth was inserted into the wound immediately. The amount of blood lost until hemostasis was reached and the time to hemostasis was recorded for each group. After the hemostatic sealing was confirmed, the incision was closed using sutures. Two weeks after the implantation, the rats were euthanized by 5% isoflurane induction followed by CO₂ inhalation. Livers with the implants were excised and fixed in 4% paraformaldehyde solution for 48 hours for histological analysis.

### Rat Liver Incision Bleeding Model

For hemostatic sealing of the deep incisional hepatic injury, the rats were anesthetized using isoflurane (4% isoflurane in oxygen) in an induction chamber. Anesthesia was maintained at 2% isoflurane using a nose cone during the surgery. A volume of 1 mL of saline was injected subcutaneously. Abdominal hair was removed, and the rats were placed over a heating pad for the duration of the surgery. The liver was exposed via a laparotomy. A laceration wound of 7 mm length and 3 mm depth was made to the liver using a #11 scalpel. Immediately after wiping off the blood using a hemostat selected from gauze, a pre-weighed 2M-LIMB, NB, or a commercial hemostatic agent was applied to the site of the lesion. The hemostat was held in place by hand without applying compression to the wound. The amount of blood lost until hemostasis was reached and the time to hemostasis was recorded for each group. The bleeding time of 2M-LIMB was only checked after 2 mins. After the surgery, the rats were euthanized by 5% isoflurane induction followed by CO₂ inhalation.

### Pig Liver Incision Bleeding Model

This experiment was approved by the University of British Columbia Animal Care Committee (Protocol #A18-0348) and performed according to the guidelines of the Canadian Council on Animal Care. All procedures were performed with the animals under general anesthesia. Female pigs weighing 40-50 kg were induced with 4% isoflurane, endotracheally intubated and mechanically ventilated at 10-12 breaths/min. Anesthesia was maintained with 1-3% isoflurane in combination with propofol (2-7 mg/kg/h) and midazolam (0.4-0.7 mg/kg/h). Long, central IV catheters were placed in both ears for drug and fluid administration. An intra-arterial catheter was placed in both the hind leg pedal arteries for invasive blood pressure measurement and these catheters were taped and bandaged in place to allow for blood collection throughout the recovery period. Temperature was maintained at 38.5-39.5° C. with a heating pad and was monitored using a rectal temperature probe. Hydration was maintained with 1.25% dextrose in isolyte solution administered intravenously 3 to 5 ml/kg/hr.

With the animal in dorsal recumbency, a laparotomy was performed. Liver lacerations that were 1.5 cm long and 1 cm deep were created using a scalpel. Plain gauze or 2M-LIMB was applied gently to the injury site without additional manual compression. Blood loss was quantified by placing pre-weighed gauze into the abdominal cavity and measuring the change in mass of the gauze. Time to hemostasis was measured. The monitoring period lasted 10 min, after which time manual compression was used to definitively stop the bleed.

### In Vivo Hemostatic Tests

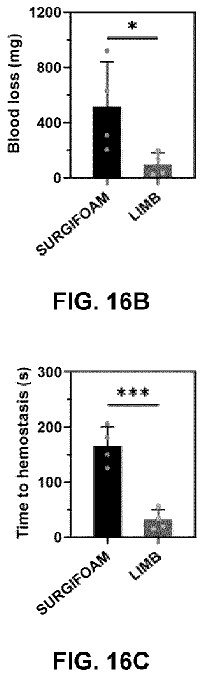

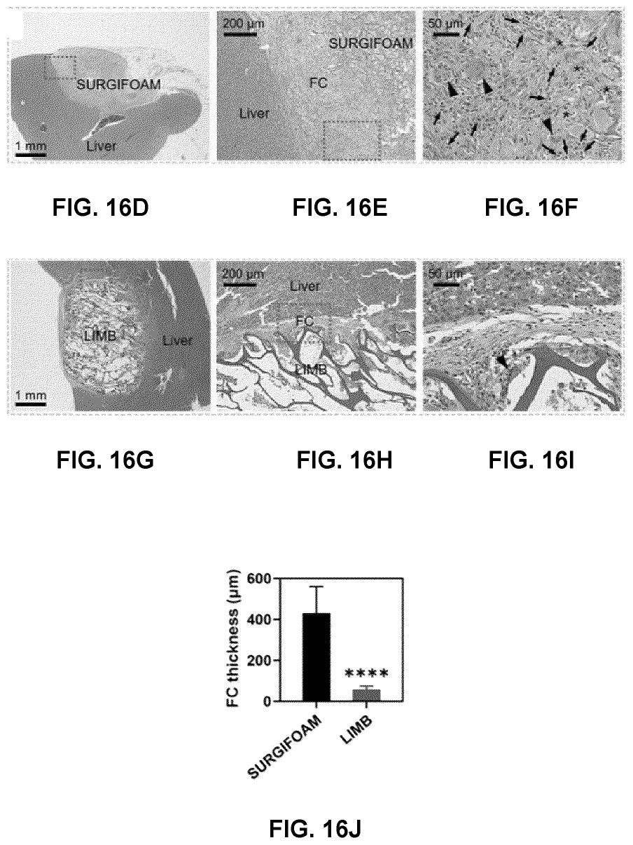

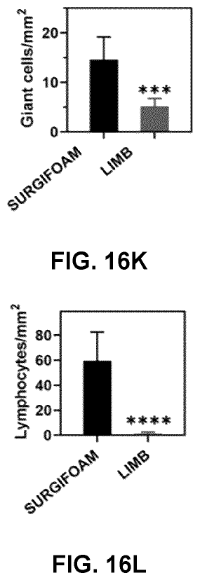

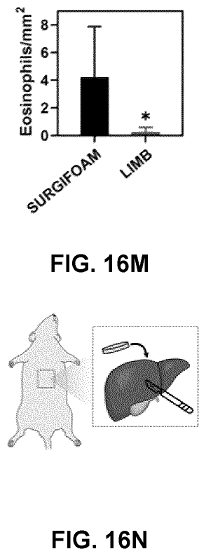

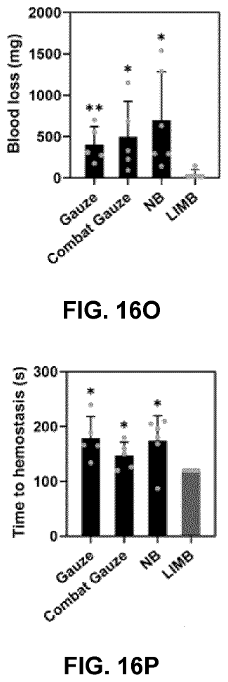

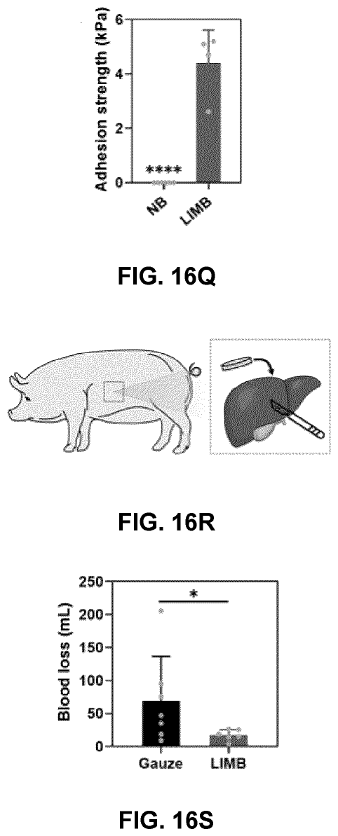

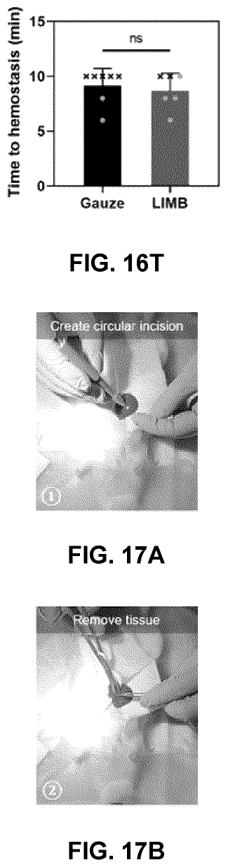

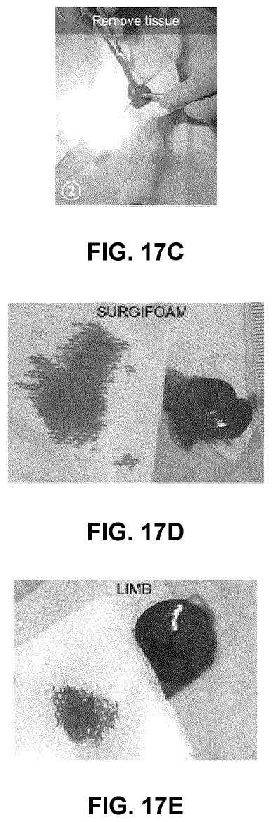

The safety and efficacy of 2M-LIMB was evaluated in different animal models with physiological relevance in comparison with commercial absorbable hemostatic gelatin sponge SURGIFOAM™ (Ethicon). Deep wounds with small entrances, for instance, caused by small arms fire limit the direct contact between hemostats and bleeding vessels. The resulting non-compressible hemorrhage has limited treatment outcomes with current hemostatic technologies. A rat liver was used as a volume defect model (4 mm in diameter and 3 mm in depth) to evaluate the efficacy of 2M-LIMB as an implantable hemostat to halt non-compressible hemorrhage (FIG. 16A and FIGS. 17A-17E). The liver defect received 2M-LIMB or SURGIFOAM™ of the same size. The hemostatic efficacy of 2M-LIMB was manifested with low blood loss (99.8 mg) and quick time to hemostasis (31.5 s). Compared to 517.1 mg and 165.8 s for SURGIFOAM™, the 2M-LIMB achieved an overall 5-fold improvement in both criteria (FIGS. 16B-16C).

In addition to the acute response, long-term biocompatibility in vivo was evaluated by examining the materials implanted for two weeks after hemostasis (FIGS. 16D-16I). Four metrics of potential immune responses were used, including the thickness of fibrotic capsule (FC), giant cells density for foreign body reactions, lymphocytes density for inflammatory responses, and eosinophils density for allergy responses. The statistical analysis substantiated a considerable advantage of 2M-LIMB over SURGIFOAM™ in all metrics measured. More specifically, 2M-LIMB induced thin FC (˜53 μm), very mild foreign body reactions, minimal inflammation, and allergy responses. In comparison, SURGIFOAM™ lead to a thick FC (˜431 μm) and a high degree of foreign body and inflammation responses. The allergy response was moderate but significantly higher than that of 2M-LIMB (FIGS. 16J-16M). Overall, 2M-LIMB demonstrated excellent utility as implantable hemostats for halting non-compressible hemorrhages.

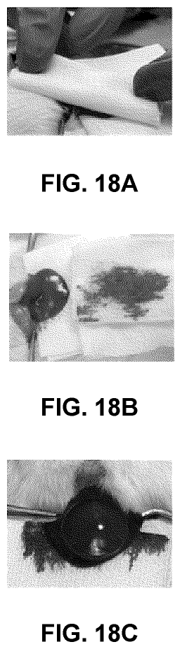

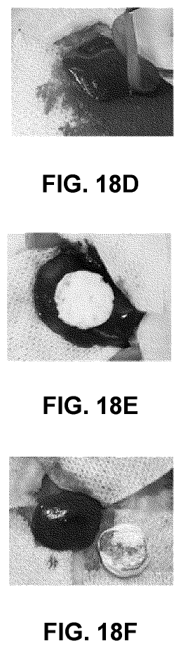

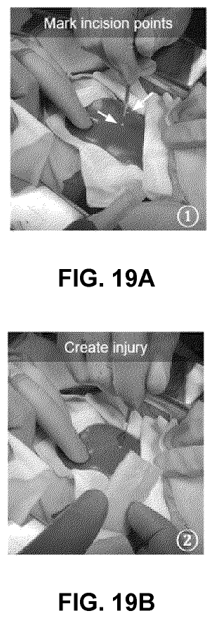

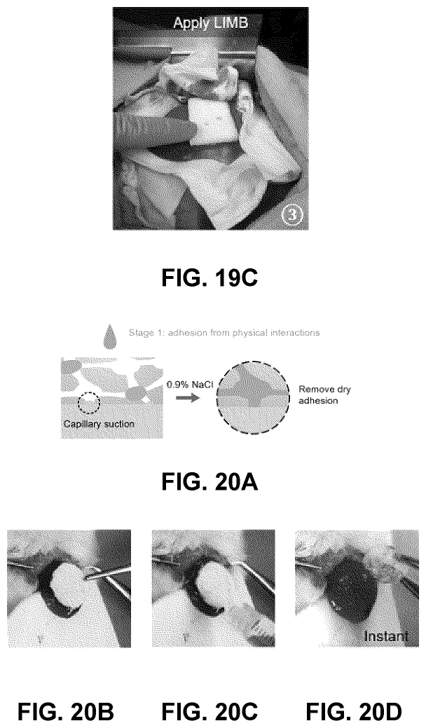

2M-LIMB was further tested for treating deep incision hemorrhage. Both rat and pig liver incision models were used. NB, standard gauze, and a commercially available Combat Gauze (QuickClot) were included for comparison. The hemostats were positioned steadily at bleeding sites and exposed to no pressure to test the pressure-insensitive hemostasis. For the rat model, an incision of 7 mm in length and 3 mm in depth was created on the liver. The resulting blood loss was significantly lower for 2M-LIMB compared to other hemostats, including NB, standard gauze, and Combat Gauze (FIGS. 16N, 16O and 18A-18F). Due to the translucent appearance, the exact time to hemostasis of 2M-LIMB was difficult to measure. All the tested 2M-LIMBs stopped the bleeding within the 120 s′ check-up point and were much quicker than the comparative products (FIG. 16P). A “pull-off” test was used to measure the adhesion strength of 2M-LIMB to the bleeding liver after 120 s. 2M-LIMB was securely attached to the wounded liver, showing a significantly higher adhesion strength compared to NB (FIG. 16Q). The formation of adhesion indicated that 2M-LIMB successfully removed the interfacial blood. For the pig model, incisions of 15 mm in length and 10 mm in depth on the liver were created (FIG. 16R and FIGS. 19A-19C). The average blood loss for gauze and 2M-LIMB were 69.5 and 17.1 mL, respectively (FIG. 16S). The time to hemostasis within a 10-minute cut-off time was also observed for both groups. Successful hemostasis events are denoted with dots and unsuccessful hemostasis with crosses in FIG. 16T. In 4 out of 6 swine injuries treated with 2M-LIMB, the bleeding stopped within 10 min, but only in 2 of 7 injuries treated with plain gauze succeeded. The successful hemostasis rate was 66.7% for 2M-LIMB and 28.6% for gauze, an over 2.3-fold improvement. The combined results showed the efficacy of 2M-LIMB in halting deep incision hemorrhages.

### LIMB Removal

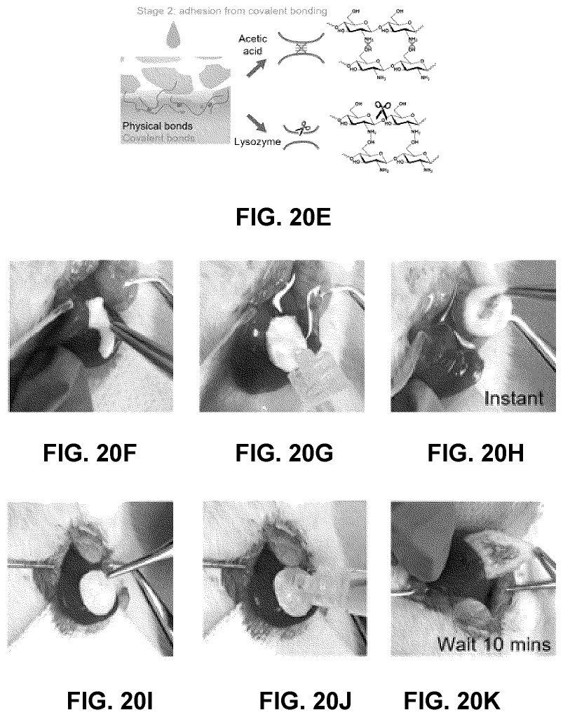

LIMB can be instantly and safely removed on-demand, which is an unmet need for existing bioadhesives. The adhesion of LIMB contains two stages. In the first stage, roughly the first two minutes following the placement, the adhesion mainly comes from the capillary suction from the dry pores of LIMB. These physical interactions can be easily disabled by wetting LIMB with saline (0.9% NaCl). It was shown that 2M-LIMB can be safely peeled off from the wounded liver immediately after wetting (FIGS. 20A-20K). Similarly, this method was also applied to repositioning procedures as they are usually decided within 1-2 minutes. It was found that 2M-LIMB can be detached from the surface easily with the wetting or be directly peeled off.

Beyond this time window, 2M-LIMB was still removable with removal agents to cleave bonds formed at the interface. The removal agents are generally needed because covalent bonds start to appear after 2 minutes due to EDC, and plateau in around 10 minutes. Once chemical bonds form, 2M-LIMB cannot be easily detached from the tissue simply by wetting. It was demonstrated that two removal agents: acetic acid solution (0.1 M) and lysozyme solution (75 mg/mL) can be used. Acetic acid can quickly disrupt those bonds and even dissolve chitosan networks at the interface and within 2M-LIMB, which are responsible for wet adhesion (note that the acetic acid solution used was at a low concentration and can be quickly neutralized with saline afterward). Alternatively, lysozyme acts like scissors to cut down the chitosan chains. After 10-minute exposure to lysozyme, the adhesion between 2M-LIMB and the wounded liver was noticeably weakened. It was observed that no rebleeding occurred after the removal of 2M-LIMB with either acetic acid or lysozyme. This phenomenon was attributed to the capacity of 2M-LIMB to promote coagulation, an advantage over existing bioadhesive sealants that only physically block the bleeding site.

### Polyethylene Glycol (PEG) Diacrylate Hydrogels

PEG was tested as an alternative to PAAm in the formation of LIMB. PEG-diacrylate (PEGDA) was used and the reagents are summarized in Table 2 below.

TABLE 2
Reagents for the formation of LIMB with PEGDA

Molecular Weight (g/mol)

| | | |
|---|---|---|
| PEGDA 20K | SIGMA | |
| SC (NaHCO₃) | S233-500 Fisher | 84.01 |
| Dibasic* | Na₂HPO₄ | 141.96 |
| Monobasic* | NaH₂PO₄ | 119.98 |
| irgacure ™ 2959 | SIGMA | |

*the molecular weight for dibasic and monobasic sodium phosphate salts are anhydrous. The molecular weight of water needs to be fractioned in if the salts contain water.

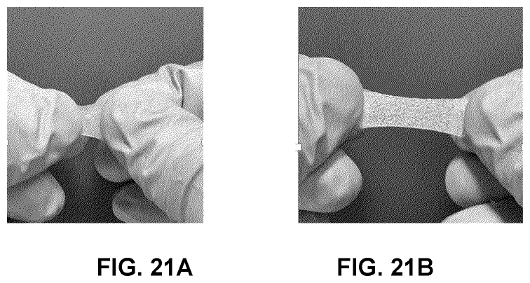

A solution was prepared at pH 8 by mixing 0.1 M dibasic and 0.1 M monobasic in a volume ratio of 50:3 to obtain a first mixture and then dissolving 1.0283 g of SC in 40 mL of the first mixture to obtain a solution with 0.306 M SC referred to as solution A. The chitosan solution was obtained as previously described by first adding 603.5 μL of pure acetic acid into 50 mL of DI water to obtain an acetic acid solution. Then, 1.25 g of chitosan were dissolved in 50 mL of the acetic acid solution. The chitosan/PEGDA solution was obtained by dissolving 0.3 g of PEGDA into 1.8 mL of chitosan in a first syringe labelled “syringe 1”. The contents of syringe 1 were mixed until a transparent homogeneous solution was obtained. Syringe 1 was stored at 4° C. An amount of 54 mg of the photoinitiator Irgacure™ 2959 (i2959) was dissolved into 6780 μL of ethanol, then 67.8 μL of the dissolution was combined with 1.18 mL of the first mixture and added into a second syringe labelled “syringe 2”. Syringes 1 and 2 (which are 10 mL syringes) were then connected using a syringe connector. Syringes 1 and 2 were degassed under vacuum for around fifteen minutes. Then, the contents of syringes 1 and 2 were mixed and the mixture was poured into a glass mold that was capped with a spacer of 1.5 mm thick PMMA sheet, size 50*80 mm and another glass mold before oxydiation could occur (i.e. before the mixture changes color). The mixture in the mold was then cured for 30 mins (15 mins on each side) with an ice bag pad until the reaction ended. The final gel composition was 1.5 wt. % chitosan and 10% PEGDA (w/v %) and demonstrated a large deformability and similar properties as the PAAm LIMB (FIGS. 21A-21B).

1. A hemostatic bioadhesive comprising:
from 0.5 to 5 w/v % of chitosan or alginate measured as a weight percent of chitosan or alginate with respect to a total dry volume of the hemostatic bioadhesive, the chitosan or the alginate being crosslinked and forming a first polymer network;
from 0.5 to 13 w/v % of polyacrylamide or polyethylene glycol measured as a weight percent of polyacrylamide or polyethylene glycol with respect to the total dry volume of the hemostatic bioadhesive, the polyacrylamide or the polyethylene glycol being crosslinked and forming a second polymer network, wherein the first polymer network and the second polymer network form a dissipative polymer matrix;
from 0 to 100 v/v % of an adhesive liquid infused in the dissipative polymer network, measured as a volume of the adhesive liquid infused with respect to a maximum infused volume of adhesive liquid;
wherein the hemostatic bioadhesives comprise interconnected pores in the dissipative polymer matrix having a size of 20 to 400 μm.

2. The hemostatic bioadhesive according to claim 1, wherein the adhesive liquid is present in a concentration of from 0 to 25 v/v %.

3. The hemostatic bioadhesive according to claim 1, wherein the hemostatic bioadhesive comprises 0.5 to 5 w/v % of chitosan.

4. The hemostatic bioadhesive according to claim 3, wherein the chitosan is present in a concentration of from 0.75 to 2.4 w/v %.

5. The hemostatic bioadhesive according to claim 1, wherein the polyacrylamide is present in a concentration of 1 to 10 w/v %.

6. The hemostatic bioadhesive according to claim 1, wherein the adhesive liquid comprises chitosan.

7. The hemostatic bioadhesive according to claim 1, wherein the adhesive liquid comprises chitosan, N-(3-Dimethylaminopropyl)-N′-ethylcarbodiimide (EDC), and N-hydroxysuccin-imide (NHS).

8. The hemostatic bioadhesive according to claim 1, further comprising at least one therapeutic agent.

9. The hemostatic bioadhesive according to claim 8, wherein the at least one therapeutic agent comprises a blood clotting agent.

10. The hemostatic bioadhesive according to claim 8, wherein the at least one therapeutic agent comprises an antimicrobial agent.

11. The hemostatic bioadhesive according to claim 1, wherein the chitosan has a degree of deacetylation of at least 65%.

12. The hemostatic bioadhesive according to claim 1, wherein the chitosan has a molecular weight of from 50 kDa to 375 kDa.

13. The hemostatic bioadhesive according to claim 1, wherein the pores have a size of from 75 to 250 μm.

14. A method of reducing or stopping a hemorrhage at a bleeding site in a subject in need thereof, the method comprising covering the bleeding site with the hemostatic bioadhesive as defined in claim 1, allowing the hemostatic bioadhesive to absorb interfacial fluids present at the bleeding site, and bonding the hemostatic bioadhesive to the bleeding site.

15. The method of claim 14, wherein the interfacial fluid comprises blood, mucus, cerebrospinal fluid, lymph fluid and/or interstitial fluid.

16. The method of claim 14, wherein the hemorrhage is a non-compressible hemorrhage and the bonding is formed without the application of external pressure.

17. Use of the hemostatic bioadhesive as defined in claim 1 for reducing or stopping a hemorrhage at a bleeding site in a subject in need thereof.

18. The use of claim 17, wherein the hemorrhage is a non-compressible hemorrhage.

19. A method of producing a hemostatic bioadhesive, the method comprising:
mixing a solution comprising acrylamide and chitosan with a gelling solution to obtain a gel, the gel comprising 0.5 to 3 w/v % of crosslinked chitosan and 0.5 to 13 w/v % of crosslinked polyacrylamide measured as weight with respect to a total dry volume of the hemostatic bioadhesive;
freeze drying the gel at a temperature of less than −2° C. to form ice crystals in the gel; and
sublimating the ice crystals to leave pores having a size of from 20 to 400 μm.

20. A method of producing a liquid-infused microstructured bioadhesive (LIMB), the method comprising:
producing the hemostatic bioadhesive as defined in claim 19; and
infusing the hemostatic bioadhesive with up to 30 v/v % of a liquid measured as a volume of the adhesive liquid with respect to a total volume of the hemostatic bioadhesive.
